(19) United States
(12) Patent Application Publication
(10) Pub. No.: US 2014/0068809 A1
(43) Pub. Date: Mar. 6, 2014

(54) STRESS RESPONSIVE EXPRESSION

(71) Applicants: Sergiy Lopato, Morphett Vale (AU); Nataliya Kovalchuk, Marryatville (AU); Peter Langridge, Teringie (AU)

(72) Inventors: Sergiy Lopato, Morphett Vale (AU); Nataliya Kovalchuk, Marryatville (AU); Peter Langridge, Teringie (AU)

(73) Assignee: AUSTRALIAN CENTRE FOR PLANT FUNCTIONAL GENOMICS PTY LTD, URRBRAE (AU)

(21) Appl. No.: 14/002,098

(22) PCT Filed: Feb. 28, 2012
(86) PCT No.: PCT/AU2012/000191

§ 371 Date: Nov. 11, 2013

(30) Foreign Application Priority Data
Feb. 28, 2011 (AU) 2011900690

(51) Int. Cl. C12N 15/82

(52) U.S. Cl. CPC C12N 15/8273
USPC 800/278; 435/320.1; 435/419; 800/298

(57) ABSTRACT

The present invention relates to the identification of transcriptional control sequences that are active in plants in response to stress. Accordingly, methods for effecting stress responsive expression of a nucleotide sequence of interest in a plant are provided, the methods including expressing the nucleotide sequence of interest operably connected to a transcriptional control sequence which is stress inducible in the plant, wherein the nucleotide sequence of interest is heterologous with respect to the transcriptional control sequence. Also provided are nucleic acid constructs including the stress inducible transcriptional control sequences, genetically modified cells including the nucleic acid constructs, and multicellular structures including one or more of the genetically modified cells.

### FIELD OF THE INVENTION

The present invention relates generally to methods and transcriptional control sequences suitable for effecting expression of a nucleotide sequence of interest in a plant. More particularly, the present invention relates to methods and transcriptional control sequences suitable for the stress responsive expression of a nucleotide sequence of interest in one or more cells of a plant.

### BACKGROUND OF THE INVENTION

Stresses such as temperature, drought, salinity, disease and pathogen attack are common factors that negatively impact on plant growth and development. To overcome these limitations, plants respond and adapt to various stresses at the physiological and biochemical levels.

For example, several families of transcription factors, such as DREB/CBF, ERF, MYK, MYB, AREB/ABF, NAC and HDZip class I and II, have been shown to be involved in the regulation of stress response in plants. The dehydration-responsive element-binding proteins (DREBs) or C-repeat-binding proteins (CBFs), for instance, are among the first discovered families of transcription factors responsible for gene regulation under conditions of low temperature and water deficiency.

The genetic manipulation of plants has been used to effect transcription of an introduced nucleotide sequence of interest either specifically or preferentially in a plant, plant part, or at a particular developmental stage of the plant. Accordingly, there is substantial interest in identifying transcriptional control sequences, such as promoters or enhancers, which specifically or preferentially direct transcription in plants, particular plant organs, tissues or cell types, or at particular developmental stages of the plant.

Expression of a heterologous nucleotide sequence in a plant is dependent upon the presence of an operably linked transcriptional control sequence which is functional within the plant. The choice of transcriptional control sequence will determine when and where within the organism the heterologous nucleotide sequence is expressed. For example, where continuous expression is desired throughout the cells of a plant, constitutive promoters are utilised. In contrast, where gene expression in response to a stimulus (such as stress) is desired, an inducible promoter may be used.

Since discovery of the role of DREB/CBF factors in the stress response, several attempts have been undertaken to genetically manipulate plants in order to demonstrate the potential of these factors to improve stress tolerance in Arabidopsis, and crop plants such as Brassica junceae, soybean, rice, wheat and other grasses. In the majority of attempts to overexpress DREB/CBF factors in plants, constitutive promoters such as the Cauliflower mosaic virus 35S promoter, rice actin 1 promoter, maize polyubiquitin promoter, and inducible promoters such as AtRd29A, HvDhn8 and maize Rab17 promoters have been used. However, in most cases strong or even moderate constitutive expression (alternatively, high basal level of the inducible promoter activity) led to different degrees of growth retardation (which subsequently led to dwarfism of the transgenic plants), delayed flowering time and smaller spikes.

In light of the above, it would be desirable to be able to increase the expression of nucleotide sequences, including those involved in stress tolerance (such as DREB/CBF factors), in a stress responsive manner, and preferably without affecting subsequent development of the plant. Therefore, isolation and characterization of stress induced transcriptional control sequences, which can serve as regulatory regions for expression of heterologous nucleotide sequences of interest in a plant, would be desirable for use in the genetic manipulation of plants.

Reference to any prior art in this specification is not, and should not be taken as, an acknowledgment or any form of suggestion that this prior art forms part of the common general knowledge in any country.

### SUMMARY OF THE INVENTION

The present invention is predicated, in part, on the isolation and characterisation of a transcriptional control sequence derived from a plant gene. Among other things, the present invention has identified that the transcriptional control sequence can effect stress responsive expression of an operably connected heterologous nucleotide sequence in a plant.

Accordingly, in a first aspect, the present invention provides a method for effecting stress responsive expression of a nucleotide sequence of interest in one or more cells of a plant, the method including expressing in the one or more cells of the plant the nucleotide sequence of interest operably connected to a transcriptional control sequence which is stress inducible in the plant, wherein the nucleotide sequence of interest is heterologous with respect to the transcriptional control sequence.

In some embodiments, the plant is a monocot plant. In some embodiments, the plant is a cereal crop plant, such as a wheat, rice or barley plant.

In some embodiments, the transcriptional control sequence includes the nucleotide sequence set forth in SEQ ID NO: 1, or a functionally active fragment or variant thereof.

In some embodiments, the transcriptional control sequence includes the nucleotide sequence set forth in SEQ ID NO: 6, or a functionally active fragment or variant thereof.

In some embodiments, the nucleotide sequence of interest includes a nucleotide sequence which, when expressed by one or more cells of a plant, improves the stress tolerance of the plant. In one embodiment, expression of the nucleotide sequence does not disturb development of the plant.

In some embodiments, the stress is cold. Accordingly, in some embodiments, the nucleotide sequence of interest includes a nucleotide sequence which, when expressed by one or more cells of a plant, improves the cold tolerance of the plant. In one embodiment, expression of the nucleotide sequence does not disturb development of the plant.

In some embodiments, the nucleotide sequence of interest includes a nucleotide sequence that encodes a DREB polypeptide. In one embodiment, the DREB polypeptide is a TaDREB3-like polypeptide.

In a second aspect, the present invention provides a nucleic acid construct including a nucleotide sequence of interest operably connected to transcriptional control sequence which is stress inducible in a plant, wherein the nucleotide sequence of interest is heterologous with respect to the transcriptional control sequence.

In some embodiments, the transcriptional control sequence is stress inducible in a monocot plant. In some embodiments, the transcriptional control sequence is stress inducible in a cereal crop plant, such as a wheat, rice or barley plant.

In some embodiments of the second aspect of the invention, the transcriptional control sequence includes the nucleotide sequence set forth in SEQ ID NO: 1, or a functionally active fragment or variant thereof.

In some embodiments of the second aspect of the invention, the transcriptional control sequence includes the nucleotide sequence set forth in SEQ ID NO: 6, or a functionally active fragment or variant thereof.

In some embodiments of the second aspect of the invention the nucleotide sequence of interest includes a nucleotide sequence which, when expressed by one or more cells of a plant, improves the stress tolerance of the plant. In one embodiment, expression of the nucleotide sequence does not disturb development of the plant.

In some embodiments the stress is cold. Accordingly, in some embodiments, the nucleotide sequence of interest includes a nucleotide sequence which, when expressed by one or more cells of a plant, improves the cold tolerance of the plant. In one embodiment, expression of the nucleotide sequence does not disturb development of the plant.

In some embodiments of the second aspect of the invention, the nucleotide sequence of interest includes a nucleotide sequence that encodes a DREB polypeptide. In one embodiment, the DREB polypeptide is a TaDREB3-like polypeptide.

In some embodiments of the second aspect of the invention, the nucleic acid construct may further include a nucleotide sequence defining a transcription terminator. In some embodiments, the nucleic acid construct includes an expression cassette including the structure:

([N]_{w}-TCS-[N]ₓ-Sol-[N]_{y}-TT-[N]_{z})

wherein:

[N]_{w} includes one or more nucleotide residues, or is absent;

TCS defines the transcriptional control sequence;

[N]ₓ includes one or more nucleotide residues, or is absent;

Sol includes the nucleotide sequence of interest that is heterologous with respect to the TCS, wherein the nucleotide sequence of interest encodes an mRNA or non-translated RNA, and is operably connected to the TCS;

[N]_{y} includes one or more nucleotide residues, or is absent;

TT includes a nucleotide sequence defining a transcription terminator; and

[N]_{z} includes one or more nucleotide residues, or is absent.

In a third aspect, the present invention provides a genetically modified cell including a nucleic acid construct of the second aspect of the invention, or a genomically integrated form thereof.

In some embodiments, the cell is a plant cell. In some embodiments, the cell is a monocot plant cell. In some embodiments, the cell is a cereal crop plant cell, such as a wheat, rice or barley plant cell.

In a fourth aspect, the present invention provides a multicellular structure including one or more cells of the third aspect of the invention.

In some embodiments, the multicellular structure includes a plant or a part, organ or tissue thereof. In some embodiments, a nucleotide sequence of interest is expressed in one or more cells of the plant or a part, organ or tissue thereof in response to stress. For example, the stress may be cold.

In some embodiments, the multicellular structure includes a monocot plant or a part, organ or tissue thereof. In some embodiments, the multicellular structure includes a cereal crop plant or a part, organ or tissue thereof, such as a wheat, rice or barley plant or a part, organ or tissue thereof.

In some embodiments of the fourth aspect of the invention, the plant or a part, organ or tissue thereof has improved stress tolerance relative to a plant or a part, organ or tissue thereof which does not include one or more cells of the third aspect of the invention. For example, the stress may be cold.

### BRIEF DESCRIPTION OF THE FIGURES

FIG. 1 shows a schematic representation of the pWRKY71-TaDREB3 nucleic acid construct used in Examples 1 and 2. The WRKY71 transcriptional control sequence is operably connected to a heterologous DREB3 nucleic acid.

FIG. 2 shows Northern blot analysis of transgene expression (TxDREB3) in 5 independent T₁ transgenic wheat lines (L1, L6, L12, L15 and L26) in Example 1. Time points in hours (h) correspond to growth of seedlings giving rise to each line at 4° C. PC: positive control.

FIG. 3 shows quantitative PCR analysis of transgene expression (TxDREB3), endogene expression (TaDREB3) and potential target gene expression (TaCor14B) in 3 independent T₁ transgenic wheat lines (L6, L12 and L15) in Example 1. Time points in hours (h) correspond to growth of seedlings giving rise to each line at 4° C.

FIG. 4 shows a schematic of the protocol used for a frost tolerance experiment in Example 1.

FIG. 5 shows the results of the frost tolerance experiment in Example 1 using T₁ transgenic wheat lines with cold inducible expression of DREB3. (A) A table showing the percent of transgenic and control plants which recovered two weeks after exposure to frost. (B) Photographs showing growth of a representative transgenic wheat line (L5) and control plants (indicated by arrows) prior to the frost tolerance experiment (upper panel), and one week after exposure to frost (lower panel).

FIG. 6 shows a schematic representation of the pCor39-TaDREB3 nucleic acid construct used in Example 2. The COR39 transcriptional control sequence is operably connected to a heterologous DREB3 nucleic acid.

FIG. 7 is a schematic showing the design (A) and conditions (B) of the frost tolerance test for barley plants in Example 2. The scheme is not drawn to scale. The conditions of the experiment provide 10-50% survival of control barley cv. Golden Promise. Time of collecting leaf samples for the Northern blot analysis is indicated with arrows.

FIG. 8 provides graphs showing expression of the Cor39 gene in various wheat tissues and activation by different stresses demonstrated by Q-PCR in Example 2. (A) Expression of the TaCor39 gene in different tissues of bread wheat in the absence of stress. (B) Expression of the TdCor39 gene in leaves of 4 week-old seedlings of durum wheat subjected to cold stress. (C) Expression of the TdCor39 gene in leaves of 4 week-old seedlings of durum wheat subjected to drought stress and following re-watering. (D) Expression of the TaCor39 in leaves of 4 week-old seedlings subjected to wounding.

FIG. 9 shows the results of analysis of transgene copy number and constitutive levels of transgene expression in T₁ barley plants used for subsequent phenotyping in Example 2. (A) Copy number of the TaDREB3 gene in T₁ barley plants measured by Q-PCR. (B) Basal levels of OsWRKY71 and TdCor39 promoter activity in transgenic barley plants measured by Q-PCR.

FIG. 10 shows graphs representing a comparison of phenotypes and grain yields of transgenic and control (WT) plants in Example 2. Results for the eight null segregants (L16-5, L2-1, L2-2, L2-7, L5-5, 15-7, L18-2, L19-1) were combined together as a second control (Null).

FIG. 11 shows photographs of control (WT) and transgenic T₁ barley plants of Example 2 two weeks before flowering. (A) Plants transformed with pWRKY-TaDREB3 construct. (B) Plants transformed with pCor39-TaDREB3 construct. Null segregants are marked with arrows.

FIG. 12 shows a graphic representation of the delay in flowering time for T₁ transgenic plants of Example 2 transformed with either the pWRKY71-TaDREB3 or pCor39-TaDREB3 constructs. Flowering time of transgenic plants was compared with the average flowering time of seven control plants, which is represented as day 0.

FIG. 13 shows the results of the frost test in Example 2 for barley plants transformed with the pWRKY71-TaDREB3 construct. (A) Survival rates for control plants (WT) and three independent transgenic lines (L2, L5 and L16). (B) Transgene activation in tested plants demonstrated by Northern blot hybridisation as compared to control (C) plants.

FIG. 14 shows the results of the frost test in Example 2 for barley plants transformed with the pCor39-TaDREB3 construct. (A) Survival rates for control plants (WT) and three independent transgenic lines (L12, L18 and L19). (B) Transgene activation in tested plants demonstrated by Northern blot hybridisation as compared to control (C) plants.

FIG. 15 shows the results of the frost test in Example 2 for barley plants transformed with the pCor39-TaDREB3 construct. (A) Survival rates for control plants (WT) and three independent transgenic lines (L5, L18 and L20). (B) Transgene activation in tested plants demonstrated by Northern blot hybridisation as compared to control (C) plants.

FIG. 16 shows a graphic representation of activation of transgene (TaDREB3—FIG. 16A) and cold-responsive downstream genes (HvCor14B—FIG. 16B, HvDhn8—FIG. 16C, and HvA22—FIG. 16D) in control (WT) and selected T₁ transgenic barley plants demonstrated by Q-PCR. a—leaf samples were collected before stress, b—leaf samples were collected after several hours of acclimation at 4° C.

FIG. 17 shows a graphic representation of the result of activation of the OsWRKY71 and TdCor39 promoters in leaf, stem and developing spike by incubation of plants at constant 4° C. as demonstrated by Q-PCR.

FIG. 18 shows the results of Q-PCR and phenotypic analysis of transgenic rice plants comprising the pWRKY71-TaDREB3 transgene. (A) Basal levels of the OsWRKY71 promoter activity in transgenic T₀ rice plants (L2 and 3) measured by Q-PCR. (B) Induction of the OsWRKY71 promoter activity by cold stress in transgenic T₁ rice plants; leaf samples were collected at 0, 1, 2 and 4 hours after plant transfer to 4° C.; M—molecular weight markers, P—positive control. (C) Phenotype characteristics of 9 week-old transgenic T₁ rice plants.

### DESCRIPTION OF THE INVENTION

Nucleotide sequences are referred to herein by a sequence identifier number (SEQ ID NO:). A summary of the sequence identifiers is provided in Table 1. A sequence listing is also provided.

TABLE 1
Summary of Sequence Identifiers

| Sequence | |
|---|---|
| Identifier | Sequence |
| SEQ ID NO: 1 | OsWRKY71 promoter nucleotide sequence |
| SEQ ID NO: 2 | OsWRKY71F primer sequence - promoter |
| | amplification |
| SEQ ID NO: 3 | OsWRKY71F primer sequence - promoter |
| | amplification |
| SEQ ID NO: 4 | TaCor39F primer sequence - TdCor39 amplification |
| SEQ ID NO: 5 | TaCor39R primer sequence - TdCor39 amplification |
| SEQ ID NO: 6 | TdCor39 promoter nucleotide sequence |
| SEQ ID NO: 7 | TdCor39F primer sequence - promoter amplification |
| SEQ ID NO: 8 | TdCor39R primer sequence - promoter amplification |
| SEQ ID NO: 9 | PF3′ primer sequence |
| SEQ ID NO: 10 | NTR5′ primer sequence |
| SEQ ID NO: 11 | HPF primer sequence - Q-PCR normalisation |
| | (barley and wheat) |
| SEQ ID NO: 12 | HPR primer sequence - Q-PCR normalisation |
| | (barley and wheat) |
| SEQ ID NO: 13 | TMBW probe sequence - TaqMan probe (barley |
| | and wheat) |
| SEQ ID NO: 14 | SPSF primer sequence - Q-PCR normalisation (rice) |
| SEQ ID NO: 15 | SPSR primer sequence - Q-PCR normalisation (rice) |
| SEQ ID NO: 16 | TMR probe sequence - TaqMan probe (rice) |
| SEQ ID NO: 17 | HygF primer sequence - Q-PCR transgene analysis |
| SEQ ID NO: 18 | HygR primer sequence - Q-PCR transgene analysis |
| SEQ ID NO: 19 | TMTr probe sequence - TaqMan probe (transgene) |
| SEQ ID NO: 20 | TaDREB3F primer sequence - endogene Q-PCR |
| SEQ ID NO: 21 | TaDREB3R primer sequence - endogene Q-PCR |
| SEQ ID NO: 22 | TxDREB3F primer sequence - transgene Q-PCR |
| SEQ ID NO: 23 | TxDREB3R primer sequence - transgene Q-PCR |
| SEQ ID NO: 24 | HvCor14BF primer sequence - cold-responsive Q-PCR |
| SEQ ID NO: 25 | HvCor14BR primer sequence - cold-responsive Q-PCR |
| SEQ ID NO: 26 | HvDhn8F primer sequence - cold-responsive Q-PCR |
| SEQ ID NO: 27 | HvDhn8R primer sequence - cold-responsive Q-PCR |
| SEQ ID NO: 28 | HvDhn5F primer sequence - cold-responsive Q-PCR |
| SEQ ID NO: 29 | HvDhn5R primer sequence - cold-responsive Q-PCR |
| SEQ ID NO: 30 | HvA22F primer sequence - cold-responsive Q-PCR |
| SEQ ID NO: 31 | HvA22R primer sequence - cold-responsive Q-PCR |

As set out above, the present invention is predicated, in part, on the identification of a transcriptional control sequence which is active in plants.

Accordingly, in a first aspect, the present invention provides a method for effecting stress responsive expression of a nucleotide sequence of interest in one or more cells of a plant, the method including expressing in the one or more cells of the plant the nucleotide sequence of interest operably connected to a transcriptional control sequence which is stress inducible in the plant, wherein the nucleotide sequence of interest is heterologous with respect to the transcriptional control sequence.

Reference herein to a plant may include seed plant species such as monocotyledonous angiosperm plants (“monocots”), dicotyledonous angiosperm plants (“dicots”) and/or gymnosperm plants.

In some embodiments, the plant is a monocot plant. In some embodiments, the plant is a cereal crop plant. As used herein, the term “cereal crop plant” may include a member of the Poaceae (grass) family that produces grain. Examples of Poaceae cereal crop plants include wheat, rice, barley, maize, millets, sorghum, rye, triticale, oats, teff, wild rice, spelt and the like. The term cereal crop plant should also be understood to include a number of non-Poaceae plant species that also produce edible grain, which are known as the pseudocereals and include, for example, amaranth, buckwheat and quinoa.

In some embodiments, the plant is a wheat plant. As referred to herein, “wheat” should be understood as a plant of the genus Triticum. Thus, the term “wheat” encompasses diploid wheat, tetraploid wheat and hexaploid wheat. In some embodiments, the wheat plant may be a cultivated species of wheat including, for example, T. aestivum, T. durum, T. monococcum or T. spelta. In some embodiments, the term “wheat” refers to wheat of the species Triticum aestivum.

In some embodiments, the plant is a rice plant. As referred to herein, “rice” should be understood to include several members of the genus Oryza, including the species Oryza sativa and Oryza glaberrima. The term “rice” thus encompasses rice cultivars such as japonica or sinica varieties, indica varieties and javonica varieties. In some embodiments, the term “rice” refers to rice of the species Oryza sativa.

In some embodiments, the plant is a barley plant. As referred to herein, “barley” includes several members of the genus Hordeum. The term “barley” encompasses cultivated barley including two-row barley (Hordeum distichum), four-row barley (Hordeum tetrastichum) and six-row barley (Hordeum vulgare). In some embodiments, barley may also refer to wild barley, (Hordeum spontaneum). In some embodiments, the term “barley” refers to barley of the species Hordeum vulgare.

As set out above, the method according to a first aspect of the present invention contemplates effecting stress responsive expression of a nucleotide sequence of interest in one or more cells of a plant.

As referred to herein, “expression” of a nucleotide sequence of interest refers to the transcription of the nucleotide sequence in one or more cells of a plant. However, this definition in no way implies that expression of the nucleotide sequence must occur in all cells of the plant.

“Stress responsive expression”, as referred to herein, should be understood to refer to an increase in the transcription of a nucleotide sequence of interest in one or more cells of the plant when the plant experiences stress. In some embodiments, the level of increase in expression of the nucleotide sequence of interest may be at least about 2 times greater as a result of stress compared to the level of expression of the nucleotide sequence of interest in the absence of stress. In some embodiments, the level of increase in expression may be at least about 3, 4 or 5 times greater than in the absence of stress. However, a person skilled in the art would understand that expression level increases greater than this (for example at least about 10 times, 100 times, 1,000 times or even 10,000 times) in the presence of stress are also contemplated by the present invention.

“Stress” as referred to herein should be understood to include any environmental condition the plant, or cells of the plant, experiences which is suboptimal for the growth and/or development of the plant or cell thereof. In some embodiments, the stress may be abiotic in nature, for example the stress may include one or more of low temperature (e.g. frost), drought, salinity, high temperature, high irradiance, and nutrient toxicities or deficiencies. In some embodiments, the stress may be biotic in nature, for example the stress may include disease or invasion by a pathogen or pest. In some embodiments, reference herein to “stress” includes environmental conditions of sufficient severity to cause visible symptoms in a plant such as loss of turgor, wilting, rolled leaves, chlorosis, growth retardation and/or death of a plant.

“Cold” as referred to herein should be understood to include any situation where the temperature in which the plant is exposed is less than the optimum temperature of growth for that plant. In some embodiments, cold may include frost which is a result of the formation of ice crystals in cells of the plant due to the temperature of the plant falling below freezing and falling below the dew point of the surrounding air. In some embodiments, cold may include temperatures in the range of less than about 10° C., less than about 9° C., less than about 8° C., less than about 7° C., less than about 6° C., less than about 5° C., less than about 4° C., less than about 3° C., less than about 2° C., less than about 1° C., about 0° C., or less than about 0° C.

“Drought” as referred to herein should be understood to include any situation wherein the amount of water available to a plant, at a physiologically appropriate level of salinity, is less than the optimum level of water for that plant. In some embodiments, drought may include a low volumetric water content (VWC) in a soil. In some embodiments, drought may include a soil VWC of less than about 10%, less than about 9%, less than about 8%, less than 7%, less than about 6%, less than 5%, less than about 4%, or less than about 3%.

In some embodiments, drought may also include other forms of osmotic stress such as wherein a relatively high volume of water is available, but the level of salinity in the water is sufficiently high to cause osmotic stress in the plant. As would be understood by a person skilled in the art, “salinity” as used herein generally refers to the level of salt in the growing environment of a plant. A salt in this regard typically includes sodium chloride, magnesium and calcium sulphates, and bicarbonates. However, the most relevant salt for a majority of cropping systems is sodium chloride.

In the method according to a first aspect of the present invention stress responsive expression of the nucleotide sequence of interest is effected by the nucleotide sequence of interest being operably connected to a transcriptional control sequence which is stress inducible in the plant.

As used herein, the term “transcriptional control sequence” should be understood as a nucleotide sequence that modulates at least the transcription of an operably connected nucleotide sequence of interest. As such, the transcriptional control sequence of the present invention may comprise any one or more of, for example, a leader, promoter, 5′ or 3′ untranslated region (UTR), enhancer or upstream activating sequence. In some embodiments, the transcriptional control sequence may comprise a promoter and/or 5′ UTR. A “promoter” as referred to herein, encompasses any nucleic acid that confers, activates or enhances expression of an operably connected nucleotide sequence of interest in a cell.

As used herein, the term “operably connected” refers to the connection of a transcriptional control sequence, such as a promoter, and a nucleotide sequence of interest in such as way as to bring the nucleotide sequence of interest under the transcriptional control of the transcriptional control sequence. For example, promoters are generally positioned 5′ (upstream) of a nucleotide sequence to be operably connected to the promoter. In the construction of heterologous transcriptional control sequence/nucleotide sequence of interest combinations, the promoter is generally positioned at a distance from the transcription start site that is approximately the same as the distance between that promoter and the gene it controls in its natural setting, i.e. the gene from which the promoter is derived. As is known in the art, some variation in this distance can be accommodated without loss of promoter function.

As set out above, the transcriptional control sequence contemplated for use in the present invention is “stress inducible”. A stress inducible transcriptional control sequence should be understood to include transcriptional control sequences which generate an increased rate and/or increased level of transcription (expression) of an operably connected nucleotide sequence of interest in a plant when the plant is exposed to stress. In some embodiments, the stress inducible transcriptional control sequence may be activated by one or more transcription factors or other polypeptides which are expressed in a plant when the plant is exposed to stress. As referred to above, when the plant is exposed to stress the rate and/or level of transcription (expression) of the nucleotide sequence of interest may be at least about 2 times greater as a result of stress compared to the level of expression of the nucleotide sequence of interest in the absence of stress.

In some embodiments, the transcriptional control sequence may generate an increased rate and/or increased level of transcription of an operably connected nucleotide sequence which is at least about 3, 4 or 5 times greater than the rate and/or level of transcription in the absence of stress. However, expression rate and/or level increases greater than this (for example at least about 10 times, 100 times, 1,000 times or even 10,000 times) in the presence of stress are also contemplated by the present invention.

Methods to determine the rate and/or level of transcription of an operably connected nucleotide sequence of interest would be known in the art. Generally, such methods include Northern blotting and/or quantitative PCR.

Furthermore, the term “stress inducible” is to be assessed in the context of a plant of interest. For example a particular transcriptional control sequence may exhibit stress inducibility in the presence of stress in a plant of interest, but need not exhibit this characteristic in all plant species to fall within the meaning of the above-referenced term for the purposes of the present invention. In some embodiments, the term “stress inducible” may also be assessed in the context of a particular tissue type. For example, a particular transcriptional control sequence may exhibit stress inducibility in a particular plant tissue of interest in the presence of stress, e.g. the leaves, but need not exhibit this characteristic in all plant tissues to fall within the meaning of the above-referenced term for the purposes of the present invention.

As set out above, in some embodiments the transcriptional control sequence of the present invention includes the nucleotide sequence set forth in SEQ ID NO: 1 or a functionally active fragment or variant thereof, or the nucleotide sequence set forth in SEQ ID NO: 6 or a functionally active fragment or variant thereof. As referred to herein, a “functionally active fragment or variant” refers to a fragment or variant of the nucleotide sequence set forth in SEQ ID NO: 1 or the nucleotide sequence set forth in SEQ ID NO: 6 which substantially retains the ability to direct expression of an operably connected nucleotide sequence of interest in one or more cells of a plant in response to stress.

“Functionally active fragments” of the transcriptional control sequence of the present invention may be of any length provided the transcriptional control sequence retains the ability to direct expression of an operably connected nucleotide sequence in response to stress. In some embodiments, a functionally active fragment may be at least about 100 nucleotides (nt), at least about 200 nt, at least about 300 nt, at least about 400 nt, at least about 500 nt, at least about 1000 nt, at least about 1500 nt or at least about 2000 nt in length. A fragment “at least about 100 nt in length” includes, for example, fragments which include about 100 or more contiguous bases from the nucleotide sequence of SEQ ID NO: 1 or SEQ ID NO: 6.

“Functionally active variants” of the transcriptional control sequence of the present invention include orthologs, mutants, synthetic variants, analogs and the like. The functionally active variants retain the capability to direct expression of an operably connected nucleotide sequence of interest. For example, the term “variant” should be considered to specifically include transcriptional control sequences from other organisms which are orthologous to SEQ ID NO: 1 or SEQ ID NO: 6; mutants of the transcriptional control sequence of SEQ ID NO: 1 or SEQ ID NO: 6; variants of SEQ ID NO: 1 or SEQ ID NO: 6 wherein one or more of the nucleotides within the sequence has been substituted, added or deleted; and analogs that contain one or more modified bases or DNA or RNA backbones modified for stability or for other reasons. “Modified” bases include, for example, tritylated bases and unusual bases such as inosine.

As will be appreciated, functionally active fragments or variants of the nucleotide sequence set forth in SEQ ID NO: 1 or SEQ ID NO: 6 may include transcriptional control sequences isolated from other plants and/or synthetic nucleotide sequences.

In some embodiments, the functionally active fragment or variant comprises at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or 100% nucleotide sequence identity to the nucleotide sequence set forth in SEQ ID NO: 1 or SEQ ID NO: 6.

When comparing nucleic acid sequences to calculate a percentage identity, the compared nucleotide sequences should be compared over a comparison window of at least about 100 nucleotide residues, at least about 200 nucleotide residues, at least about 500 nucleotide residues, at least about 1000 nucleotide residues, at least about 1500 nucleotide residues, at least about 2000 nucleotide residues, or over the full length of SEQ ID NO: 1 or SEQ ID NO: 6. The comparison window may comprise additions or deletions (i.e. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as, for example, disclosed by Altschul et al. 1997 (Nucl. Acids Res. 25: 3389-3402). Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., 2000, Trends Genet., 16: 276-277), and the GGSEARCH program (available at fasta.bioch.virginia.edu/fasta_www2/fasta_www.cgi?rm=compare&pgm=gnw) which is part of the FASTA package (Pearson W and Lipman D, 1988, Proc. Natl. Acad. Sci. USA, 85: 2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998).

In some embodiments, the functionally active fragment or variant comprises a nucleic acid molecule which hybridises to a nucleic acid molecule defining a transcriptional control sequence of the present invention under stringent conditions. In some embodiments, the functionally active fragment or variant comprises a nucleic acid molecule which hybridises to a nucleic acid molecule comprising the nucleotide sequence set forth in SEQ ID NO: 1 or SEQ ID NO: 6 under stringent conditions.

As used herein, “stringent” hybridisation conditions will be those in which the salt concentration is less than about 1.5 M Na ion, typically about 0.01 to 1.0 M Na ion concentration (or other salts) at pH 7.0 to 8.3 and the temperature is at least 30° C. Stringent conditions may also be achieved with the addition of destabilising agents such as formamide. In some embodiments, stringent hybridisation conditions may be low stringency conditions, medium stringency conditions or high stringency conditions. Exemplary low stringency conditions include hybridisation with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridisation in 40 to 45% formamide, 1.0 M NaCl, 1% SDS at 37° C., and a wash in 0.5× to 1×SSC at 55 to 60° C. Exemplary high stringency conditions include hybridisation in 50% formamide, 1 M NaCl, 1% SDS at 37° C., and a wash in 0.1×SSC at 60 to 65° C. Optionally, wash buffers may comprise about 0.1% to about 1% SDS. Duration of hybridisation is generally less than about 24 hours, usually about 4 to about 12 hours.

Specificity of hybridisation is also a function of post-hybridisation washes, with the critical factors being the ionic strength and temperature of the final wash solution. For DNA-DNA hybrids, the Tₘ can be approximated from the equation of Meinkoth and Wahl (Anal. Biochem. 138: 267-284, 1984), i.e. Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridisation solution, and L is the length of the hybrid in base pairs. The Tₘ is the temperature (under defined ionic strength and pH) at which 50% of a complementary target sequence hybridises to a perfectly matched probe. Tₘ is reduced by about 1° C. for each 1% of mismatching; thus, Tₘ, hybridisation, and/or wash conditions can be adjusted to hybridise to sequences of different degrees of complementarity. For example, sequences with ≧90% identity can be hybridised by decreasing the Tₘ by about 10° C. Generally, stringent conditions are selected to be about 5° C. lower than the thermal melting point (Tₘ) for the specific sequence and its complement at a defined ionic strength and pH. However, high stringency conditions can utilise a hybridisation and/or wash at, for example, 1, 2, 3, or 4° C. lower than the thermal melting point (Tₘ); medium stringency conditions can utilise a hybridisation and/or wash at, for example, 6, 7, 8, 9, or 10° C. lower than the thermal melting point (Tₘ); low stringency conditions can utilise a hybridisation and/or wash at, for example, 11, 12, 13, 14, 15, or 20° C. lower than the thermal melting point (Tₘ). Using the equation, hybridisation and wash compositions, and desired Tₘ, those of ordinary skill will understand that variations in the stringency of hybridisation and/or wash solutions are inherently described. If the desired degree of mismatching results in a Tₘ of less than 45° C. (aqueous solution) or 32° C. (formamide solution), the SSC concentration may be increased so that a higher temperature can be used. An extensive guide to the hybridisation of nucleic acids is found in Tijssen (Laboratory Techniques in Biochemistry and Molecular Biology-Hybridisation with Nucleic Acid Probes, Pt I, Chapter 2, Elsevier, New York, 1993), Ausubel et al., eds. (Current Protocols in Molecular Biology, Chapter 2, Greene Publishing and Wiley-Interscience, New York, 1995) and Sambrook et al (Molecular Cloning: A Laboratory Manual, 2^{nd} ed., Cold Spring Harbor Laboratory Press, Plainview, N.Y., 1989).

In some embodiments, the nucleotide sequence of interest comprises a nucleotide sequence which, when expressed by one or more cells of a plant, improves the stress tolerance of the plant.

“Stress tolerance” as referred to herein refers to any trait in the plant which allows the plant to survive, recover and/or reproduce during or after experiencing stress. Measures of stress tolerance may include, for example, the ability of a plant to continue to grow, reproduce or yield during or after an episode of stress; the rate or frequency of recovery of plants after an episode of stress; the extent of any yield penalty for a plant after experiencing an episode of stress; the water use efficiency of a plant; and the like. “Improvement” in the stress tolerance of a plant should be seen as any increase in the ability of a plant to survive, recover or reproduce during or after experiencing stress. For example, “improved” stress tolerance of a plant may include an increased ability of a plant to continue to grow, reproduce or yield during or after an episode of stress; an increased rate or frequency of recovery of plants after an episode of stress; a decrease in or amelioration of any yield penalty associated with an episode of stress; increased water use efficiency of a plant; and the like.

As indicated above, constitutive expression of various endogenous genes can lead to an effect on the development of the plant, such as retardation in growth of the plant. Accordingly, in some embodiments of the various aspects of the present invention, the nucleotide sequence of interest comprises a nucleotide sequence which, when expressed by one or more cells of a plant, may improve the stress tolerance of the plant without disturbing development of the plant. Development of a plant will typically be assessed through a phenotypic analysis of characteristics of the plant. Such developmental characteristics include, but are not limited to, plant height, leaf length, tiller number at flowering, flowering time, spike number, main spike length, grain weight per plant, spikelet number per spike, grain number per spike, and grain weight per 1 or 100 grains.

In some embodiments, the stress is cold. Accordingly, in some embodiments, the nucleotide sequence of interest comprises a nucleotide sequence which, when expressed by one or more cells of a plant, improves the cold tolerance of the plant. In one embodiment, expression of the nucleotide sequence does not disturb development of the plant. Development of the plant can be assessed as described above.

As would be understood by one of skill in the art, the nucleotide sequence of interest, which is placed under the regulatory control of the transcriptional control sequence of the present invention, may be any nucleotide sequence which improves the stress tolerance of the plant. Examples include, but are not limited to, genes encoding transcription factors such as DREB/CBF factors, MYC factors, MYB, factors, bZip factors, ERF factors, WRKY factors, MADS factors, NAC factors etc.; genes encoding protein kinases, which are activated or transcriptionally up-regulated under stress, such as SAPKs, receptor kinases, MAP kinases, and the like; genes encoding phosphatases related to stress responses such as ZmPP2C, type 1 inositol 5-phosphatase and the like; stress inducible genes which protect cell integrity (e.g. membrane stability, chloroplast/chlorophyll stability, correct protein folding and protein stability, and the like) such as LEA, DHNs, COR, RD, LT and RAB and the like; genes encoding water channels such as aquaporins, PIPs, TIPs and NIPs; genes encoding stomata opening regulators such as AtMRP4, a guard cell plasma membrane ABCC-type ABC transporter; genes responsible for sugar metabolism such as trehalose-6-phosphate synthase (TPS) and trehalose-6-phosphate phosphatase (TPP), ABA2 (or GLUCOSE INSENSITIVE 1 [GIN1]) encoding a short-chain dehydrogenase/reductase; genes delaying stress-induced leaf senescence such as senescence associated receptor protein kinase (SARK), a gene encoding a calcium/calmodulin-regulated receptor protein kinase; calcineurin B-like proteins (CBLs); ice re-crystallisation inhibition (IRI)-like protein coding genes; and genes encoding apoplast antifreeze proteins such as chitinases, glucanases, and thaumatin-like proteins.

In some embodiments, the nucleotide sequence of interest encodes a DREB polypeptide. The dehydration-responsive element-binding proteins (DREBs) or C-repeat-binding proteins (CBFs) are among the first discovered families of transcription factors responsible for gene regulation under conditions of water deficiency.

In some embodiments, a “DREB polypeptide” as referred to herein, may comprise an AP2 domain. In some embodiments, the DREB polypeptide may comprise a single AP2 domain. The AP2 protein domain is described in detail under pfam accession number PF00847. As referred to herein, the term “dehydration-responsive element-binding proteins” or “DREB” may also encompass a C-repeat-binding protein or CBF.

Examples of DREB/CBF polypeptides include polypeptides having the following NCBI protein database accession numbers:

from Triticum aestivum—ABC86563; ABC86564; ABK55389; ABK55388; ABK55387; ABK55386; ABK55385; ABK55384; ABK55383; ABK55382; ABK55381; ABK55380; ABK55379; ABK55378; ABK55377; ABK55376; ABK55375; ABK55374; ABK55373; ABK55372; ABW87011; ABK55390; ABK55389; ABK55388; ABK55387; ABK55386; ABK55385; ABK55384; ABK55383; ABK55382; ABK55381; ABK55380; ABK55379; ABK55377; ABK55376; ABK55375; ABK55374; ABK55373; ABK55372; ABK55371; ABK55370; ABK55369; ABK55368; ABK55367; ABK55366; ABK55365; ABK55364; ABK55363; ABK55362; ABK55361; ABK55360; ABK55359; ABK55358; ABK55357; ABK55356; ABK55355; ABK55354; AAY32564; AAY32563; AAY32562; AAY32561; AAY32560; AAY32558; AAY32557; AAY32556; AAY32555; AAY32554; AAY32553; AAY32552; AAY32551; AAX28966; AAX28965; AAX28963; AAX28962; AAX28961; ACK99532; ACB69508; ACB69507; ACB69506; ACB69505; ACB69504; ACB69503; BAD66926; BAD66925; ABB90544; ABA08426; ABA08425; ABA08424; AAX13287; AAX13285; AAX13287; AAX13285; AAX13289; AAX13289; AAX13289; AAX13287; AAX13286; AAX13285; AAX13284; AAX13283; AAX13282; AAX13279; AAX13278; AAX13277; AAR05861; ABB84399; AAX28964; ABW87014; AAX13274

from Triticum monococcum—ABW87013; ABW87012; ABW87011; ABK55390; AAY32550; AAX28967;

from Aegilops speltoides subsp. Speltoides—ACO35591; ACO35590; ACO35589; ACO35588; ACO35587; ACO35586; ACO35585; ACO35584; ACO35583; AAY25517;

from Hordeum vulgare subsp. Vulgare—AAG59618; ABA25897; ABA25896; AAZ99830; AAZ99829; ACC63523; ABA25904; ABA01494; ABA01493; ABA01492; ABA01491; AAX28957; AAX28956; AAX28955; AAX28954; AAX28953; AAX28952; AAX28950; AAX28949; AAX28948; AAX23718; AAX23714; AAX23707; AAX23704; AAX23701; AAX23698; AAX23696; AAX23692; AAX23688; AAX23684; AAX23683; ABF18984; ABF18983; ABF18982; AAX28951; AAX23720; AAX23719; AAX23717; AAX23716; AAX23715; AAX23713; AAX23712; AAX23710; AAX23709; AAX23708; AAX23706; AAX23705; AAX23703; AAX23702; AAX23700; AAX23699; AAX23697; AAX23695; AAX23694; AAX23693; AAX23691; AAX23690; AAX23689; AAX23687; AAX23686; AAX23685; AAX19267; AAX19266

from Arabidopsis thaliana—NP_{—}849340; NP_{—}564496; NP_{—}181551; NP_{—}177844; NP_{—}001031837; NP_{—}567719; NP_{—}563624; NP_{—}196160; NP_{—}201318; NP_{—}191319; NP_{—}181186; NP_{—}181368; NP_{—}172721; NP_{—}176620; NP_{—}565609; NP_{—}172723; NP_{—}001077764; NP_{—}181566; NP_{—}177681; NP_{—}173355; NP_{—}680184; NP_{—}567867; NP_{—}567721; NP_{—}567720; NP_{—}565929; NP_{—}564468; NP_{—}200015; NP_{—}200012; NP_{—}201520; NP_{—}197953; NP_{—}196720; NP_{—}197346; NP_{—}193098; NP_{—}195688; NP_{—}193408; NP_{—}195408; NP_{—}194543; NP_{—}195006; NP_{—}191608; NP_{—}190595; NP_{—}187713; NP_{—}179915; NP_{—}181113; NP_{—}179810; NP_{—}182021; NP_{—}177887; NP_{—}177631; NP_{—}176491; NP_{—}173695; NP_{—}175104; NP_{—}173609; NP_{—}177931; NP_{—}174636; NP_{—}177307; NP_{—}177301; AAP13384; AAS00621; AAO39764; AAP92125; AAL40870; AAG59619; AAN85707; NP_{—}181186; AAX57275; Q3T5N4; Q0JQF7; Q9LWV3; Q6J1A5; Q64MA1; AAP83325; AAP83323; AAP83324; AAP83322; AAP83321; AAN02487; AAN02488; AAN02486

In some embodiments, the DREB polypeptide is a TaDREB3-like polypeptide.

A TaDREB3-like polypeptide, as referred to herein, should be understood as any DREB polypeptide which exhibits at least about 60%, at least about 65%, at least about 70%, at least about 75%, at least about 80%, at least about 82%, at least about 84%, at least about 86%, at least about 88%, at least about 90%, at least about 92%, at least about 94%, at least about 96%, at least about 98%, at least about 99%, or 100% sequence identity to NCBI protein accession number ABC86564 and/or CRT/DRE binding factor 5 (AAY32551; Miller et al., 2006, Mol. Genet. Genomics 275(2), 193-203). When comparing amino acid sequences to calculate a percentage identity, the compared sequences should be compared over a comparison window of at least about 50 amino acid residues, at least about 100 amino acid residues, at least about 150 amino acid residues, or over the full length of ABC86564 and/or AAY32551. The comparison window may comprise additions or deletions (ie. gaps) of about 20% or less as compared to the reference sequence (which does not comprise additions or deletions) for optimal alignment of the two sequences. Optimal alignment of sequences for aligning a comparison window may be conducted by computerised implementations of algorithms such as the BLAST family of programs as hereinbefore described.

In some embodiments, the TaDREB3-like polypeptide comprises a polypeptide encoded by an mRNA comprising the nucleotide sequence set forth in NCBI accession number DQ353853.

In light of the above, in some embodiments, the present invention provides a method for improving the stress tolerance of a plant, the method including expressing a nucleotide sequence of interest, which when expressed by one or more cells of the plant improves the stress tolerance of the plant, operably connected to a stress inducible transcriptional control sequence, wherein the nucleotide sequence of interest is heterologous with respect to the transcriptional control sequence.

As set out above, the present invention contemplates expression of a nucleotide sequence of interest under the control of a stress inducible transcriptional control sequence. In some embodiments, this is effected by introducing into the plant a nucleic acid which comprises a nucleotide sequence of interest operably connected to a stress inducible transcriptional control sequence.

The nucleic acid molecule may be introduced into the plant via any method known in the art. For example, an explant or cultured plant tissue may be transformed with a nucleic acid molecule, wherein the explant or cultured plant tissue is subsequently regenerated into a mature plant including the nucleic acid molecule; a nucleic acid may be directly transformed into a plant seed, either stably or transiently; a nucleic acid may be introduced into a seed via plant breeding using a parent plant that carries the nucleic acid molecule; and the like.

In some embodiments, the nucleic acid molecule is introduced into a plant cell via transformation. Plants may be transformed using any method known in the art that is appropriate for the particular plant species. Common methods include Agrobacterium-mediated transformation, microprojectile bombardment based transformation methods and direct DNA uptake based methods. Roa-Rodriguez et al. (Agrobacterium-mediated transformation of plants, 3^{rd} Ed. CAMBIA Intellectual Property Resource, Canberra, Australia, 2003) review a wide array of suitable Agrobacterium-mediated plant transformation methods for a wide range of plant species. Other bacterial-mediated plant transformation methods may also be utilised, for example, see Broothaerts et al., 2005 (Nature 433: 629-633). Microprojectile bombardment may also be used to transform plant tissue and methods for the transformation of plants, particularly cereal plants, are reviewed by Casas et al., 1995 (Plant Breeding Rev. 13: 235-264). Examples of direct DNA uptake transformation protocols such as protoplast transformation and electroporation are described in detail in Galbraith et al. (eds.), Methods in Cell Biology Vol. 50, Academic Press, San Diego, 1995). In addition to the methods mentioned above, a range of other transformation protocols may also be used. These include infiltration, electroporation of cells and tissues, electroporation of embryos, microinjection, pollen-tube pathway-, silicon carbide- and liposome mediated transformation. Methods such as these are reviewed by Rakoczy-Trojanowska, 2002 (Cell. Mol. Biol. Lett. 7: 849-858). A range of other plant transformation methods may also be evident to those of skill in the art and, accordingly, the present invention should not be considered in any way limited to the particular plant transformation methods exemplified above.

As set out above, the transcriptional control sequence of the present invention is introduced into a plant cell such that the nucleotide sequence of interest is operably connected to the transcriptional control sequence. The present invention contemplates any method to effect this. For example, a nucleotide sequence of interest may be incorporated into the nucleic acid molecule that comprises the transcriptional control sequence, and be operably connected thereto. In this way, the nucleotide sequence of interest and transcriptional control sequence are both introduced into the plant. Alternatively, the nucleic acid sequence of the present invention may be inserted into the plant genome such that it is placed in operable connection with an endogenous nucleic acid sequence. As would be recognised by one of skill in the art, the insertion of the transcriptional control sequence into the plant genome may be either by non-site specific insertion using standard transformation vectors and protocols, or by site-specific insertion, for example, as described in Terada et al., 2002 (Nat. Biotechnol. 20: 1030-1034).

The present invention also contemplates expression of a nucleotide sequence of interest which is “heterologous with respect to the transcriptional control sequence”. A nucleotide sequence which is “heterologous with respect to the transcriptional control sequence” should be understood to include any nucleotide sequence other than that which is operably connected to the transcriptional control sequence in its natural state. For example, in embodiments where the transcriptional control sequence includes the nucleotide sequence set forth in SEQ ID NO: 1, in its natural state SEQ ID NO: 1 is operably connected to the WRKY71 transcription factor gene in rice. Details of the WRKY71 gene and its encoded protein may be accessed from the Genbank database at the National Center for Biotechnology Information (www.ncbi.nlm.nih.gov). For example, representative Genbank Accession Numbers for WRKY71 include AB190817 (representing the complete transcribed nucleotide sequence), BK005074 (representing the complete coding sequence and intervening intron sequences), and BAF80893 (representing the amino acid sequence of the WRKY71 protein). Accordingly, in this example, any nucleotide sequence other than a nucleotide sequence set forth in Genbank Accession Numbers AB190817 and BK005074 should be considered heterologous with respect to SEQ ID NO: 1.

In embodiments where the transcriptional control sequence includes the nucleotide sequence set forth in SEQ ID NO: 6, in its natural state SEQ ID NO: 6 is operably connected to the Cor39 gene in wheat. Representative Genbank Accession Numbers for Cor39 include AF058794 (representing the complete transcribed nucleotide sequence) and AAC14297 (representing the amino acid sequence of the Cor39 protein). Accordingly, in this example, any nucleotide sequence other than a nucleotide sequence set forth in Genbank Accession No. AF058794 should be considered heterologous with respect to SEQ ID NO: 6.

As would be recognised by one of skill in the art a sequence that is “heterologous with respect to the transcriptional control sequence”, including a sequence which is “heterologous with respect to SEQ ID NO: 1” or which is “heterologous with respect to SEQ ID NO: 6”, may be derived from the same organism or a different organism from which the transcriptional control sequence, SEQ ID NO: 1, or SEQ ID NO: 6, respectively, is derived.

In a second aspect, the present invention also provides a nucleic acid construct including a nucleotide sequence of interest operably connected to a transcriptional control sequence which is stress inducible in a plant, wherein the transcriptional control sequence is heterologous with respect to the transcriptional control sequence.

The nucleic acid construct of the second aspect of the present invention may comprise any polyribonucleotide or polydeoxyribonucleotide, which may be unmodified RNA or DNA or modified RNA or DNA. For example, the nucleic acid construct may comprise single- and/or double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the nucleic acid construct may comprise triple-stranded regions comprising RNA or DNA or both RNA and DNA. The nucleic acid construct may also comprise one or more modified bases or DNA or RNA backbones modified for stability or for other reasons. A variety of modifications can be made to DNA and RNA; thus the term “nucleic acid construct” embraces chemically, enzymatically, or metabolically modified forms.

In some embodiments, the nucleic acid construct comprises DNA. Accordingly, the nucleic acid construct may comprise, for example, a linear DNA molecule, a plasmid, a transposon, a cosmid, an artificial chromosome and the like. Furthermore, the nucleic acid construct may be a separate nucleic acid molecule or may be a part of a larger nucleic acid molecule.

In some embodiments of the second aspect of the present invention, the stress inducible transcriptional control sequence may be as hereinbefore described.

In some embodiments, the transcriptional control sequence is stress inducible in a monocot plant as hereinbefore described. For example, in some embodiments the transcriptional control sequence is stress inducible in a cereal crop plant, such as a wheat, rice or barley plant as hereinbefore described.

In some embodiments, the transcriptional control sequence includes the nucleotide sequence set forth in SEQ ID NO: 1, or a functionally active fragment of variant thereof, as hereinbefore described.

In some embodiments, the transcriptional control sequence includes the nucleotide sequence set forth in SEQ ID NO: 6, or a functionally active fragment of variant thereof, as hereinbefore described.

In some embodiments, the nucleotide sequence of interest includes a nucleotide sequence which, when expressed by one or more cells of a plant, improves the stress tolerance of the plant as hereinbefore described.

As indicated above, in some embodiments the nucleotide sequence of interest comprises a nucleotide sequence which, when expressed by one or more cells of a plant, may improve the stress tolerance of the plant without disturbing development of the plant. Development of a plant can be assessed through a phenotypic analysis of characteristics of the plant as hereinbefore described.

In some embodiments, the stress is cold. Accordingly, in some embodiments, the nucleotide sequence of interest includes a nucleotide sequence which, when expressed by one or more cells of a plant, improves the cold tolerance of the plant as hereinbefore described. In one embodiment, expression of the nucleotide sequence does not disturb development of the plant. Development of the plant can be assessed as described above.

In some embodiments, the nucleotide sequence of interest encodes a DREB polypeptide as hereinbefore described. In some embodiments, the DREB polypeptide is a TaDREB3-like polypeptide as hereinbefore described.

In some embodiments, the nucleic acid construct may further include a nucleotide sequence defining a transcription terminator. The term “transcription terminator” or “terminator” refers to a DNA sequence at the end of a transcriptional unit which signals termination of transcription. Terminators are generally 3′-non-translated DNA sequences and may contain a polyadenylation signal, which facilitates the addition of polyadenylate sequences to the 3′-end of a primary transcript. As with promoter sequences, the terminator may be any terminator sequence which is operable in the cells, tissues or organs in which it is intended to be used. Examples of suitable terminator sequences which may be useful in plant cells include: the nopaline synthase (nos) terminator, the CaMV 35S terminator, the octopine synthase (ocs) terminator, potato proteinase inhibitor gene (pin) terminators, such as the pinII and pinIII terminators and the like.

In some embodiments of the second aspect of the present invention, the nucleic acid construct may include an expression cassette including the structure:

([N]_{w}-TCS-[N]ₓ-Sol-[N]_{y}-TT-[N]_{z})

wherein:

[N]_{w} includes one or more nucleotide residues, or is absent;

TCS defines the transcriptional control sequence;

[N]ₓ includes one or more nucleotide residues, or is absent;

Sol includes the nucleotide sequence of interest that is heterologous with respect to the TCS, wherein the nucleotide sequence of interest encodes an mRNA or non-translated RNA, and is operably connected to the TCS;

[N]_{y} includes one or more nucleotide residues, or is absent;

TT includes a nucleotide sequence defining a transcription terminator; and

[N]_{z} includes one or more nucleotide residues, or is absent.

The nucleic acid construct of the present invention may further include other nucleotide sequences as desired. For example, the nucleic acid construct may include an origin of replication for one or more hosts; a selectable marker gene which is active in one or more hosts; or the like.

As used herein, the term “selectable marker gene” includes any gene that confers a phenotype on a cell, in which it is expressed, to facilitate the identification and/or selection of cells which are transfected or transformed with a nucleic acid construct of the invention. A range of nucleotide sequences encoding suitable selectable markers are known in the art. Exemplary nucleotide sequences that encode selectable markers include: antibiotic resistance genes such as ampicillin-resistance genes, tetracycline-resistance genes, kanamycin-resistance genes, the AURI-C gene which confers resistance to the antibiotic aureobasidin A, neomycin phosphotransferase genes (e.g. nptI and nptII) and hygromycin phosphotransferase genes (e.g. hpt); herbicide resistance genes including glufosinate, phosphinothricin or bialaphos resistance genes such as phosphinothricin acetyl transferase-encoding genes (e.g. bar), glyphosate resistance genes including 3-enoyl pyruvyl shikimate 5-phosphate synthase-encoding genes (e.g. aroA), bromyxnil resistance genes including bromyxnil nitrilase-encoding genes, sulfonamide resistance genes including dihydropterate synthase-encoding genes (e.g. sul) and sulfonylurea resistance genes including acetolactate synthase-encoding genes; enzyme-encoding reporter genes such as GUS and chloramphenicolacetyltransferase (CAT) encoding genes; fluorescent reporter genes such as the green fluorescent protein-encoding gene; and luminescence-based reporter genes such as the luciferase gene, amongst others.

The nucleic acid constructs described herein may further include nucleotide sequences intended for the maintenance and/or replication of the construct in prokaryotes or eukaryotes and/or the integration of the construct or a part thereof into the genome of a eukaryotic or prokaryotic cell.

In some embodiments, the nucleic acid construct of the present invention is adapted to be at least partially transferred into a plant cell via Agrobacterium-mediated transformation. Accordingly, in some embodiments, the nucleic acid construct comprises left and/or right T-DNA border sequences. Suitable T-DNA border sequences would be readily ascertained by one of skill in the art. However, the term “T-DNA border sequences” should be understood to include, for example, any substantially homologous and substantially directly repeated nucleotide sequences that delimit a nucleic acid molecule that is transferred from an Agrobacterium sp. cell into a plant cell susceptible to Agrobacterium-mediated transformation. By way of example, reference is made to the paper of Peralta and Ream (Proc. Natl. Acad. Sci. USA, 82(15): 5112-5116, 1985) and the review of Gelvin (Microbiology and Molecular Biology Reviews, 67(1): 16-37, 2003).

In some embodiments, the present invention also contemplates any suitable modifications to the nucleic acid construct which facilitate bacterial mediated insertion into a plant cell via bacteria other than Agrobacterium sp., for example, as described in Broothaerts et al., 2005 (supra).

Those skilled in the art will be aware of how to produce the constructs described herein, and of the requirements for obtaining the expression thereof, when so desired, in a specific cell or cell-type under the conditions desired. In particular, it will be known to those skilled in the art that the genetic manipulations required to perform the present invention may require the propagation of a nucleic acid construct described herein or a derivative thereof in a prokaryotic cell such as an E. coli cell or a plant cell or an animal cell. Exemplary methods for cloning nucleic acid molecules are described in Sambrook et al. (Molecular Cloning: A Laboratory Manual, Cold Spring Harbor Laboratory Press, New York, 2000).

In a third aspect, the present invention provides a genetically modified cell including a nucleic acid construct of the second aspect of the invention, or a genomically integrated form thereof.

As referred to herein, a “genetically modified cell” includes any cell having a non-naturally occurring and/or introduced nucleic acid. Generally, in the case of the cells of the third aspect of the present invention, the introduced and/or non-naturally occurring nucleic acid includes a nucleic acid construct of the second aspect of the invention.

Cells of the third aspect of the invention may be transformed cells which contain the nucleic acid construct of the second aspect of the invention, or a genomically integrated form thereof, or progeny of such transformed cells which retain the construct or a genomically integrated form thereof.

As set out above, the nucleic acid construct may be maintained in the cell as a nucleic acid molecule, as an autonomously replicating genetic element (eg. a plasmid, cosmid, artificial chromosome or the like) or it may be integrated into the genomic DNA of the cell.

As used herein, the term “genomic DNA” should be understood in its broadest context to include any and all endogenous DNA that makes up the genetic complement of a cell. As such, the genomic DNA of a cell should be understood to include chromosomes, mitochondrial DNA, plastid DNA, chloroplast DNA, endogenous plasmid DNA and the like. As such, the term “genomically integrated” contemplates chromosomal integration, mitochondrial DNA integration, plastid DNA integration, chloroplast DNA integration, endogenous plasmid integration, and the like. The “genomically integrated form” of the construct may be all or part of the construct.

The cells contemplated by the third aspect of the present invention include any prokaryotic or eukaryotic cell. In some embodiments, the cell is a plant cell. In some embodiments the cell is a monocot plant cell. In some embodiments the cell is a cereal crop plant cell, for example a wheat, rice or barley plant cell as hereinbefore described.

In some embodiments, the cell may also include a prokaryotic cell. For example, the prokaryotic cell may include an Agrobacterium sp. cell (or other bacterial cell), which carries the nucleic acid construct and which may, for example, be used to transform a plant. In some embodiments, the prokaryotic cell may be a cell used in the construction or cloning of the nucleic acid construct (e.g. an E. coli cell).

In a fourth aspect, the present invention provides a multicellular structure including one or more cells of the third aspect of the invention.

In some embodiments, the multicellular structure comprises a plant or a part, organ or tissue thereof. As referred to herein, “a plant or a part, organ or tissue thereof” should be understood to specifically include a whole plant; a plant tissue; a plant organ; a plant part; a plant embryo; and cultured plant tissue such as a callus or suspension culture.

In some embodiments of the fourth aspect of the present invention, a nucleotide sequence of interest is expressed in one or more cells of the plant or a part, organ or tissue thereof in response to stress.

In some embodiments, the multicellular structure includes a monocot plant or a part, organ or tissue thereof. In some embodiments the multicellular structure includes a cereal crop plant or a part, organ or tissue thereof. For example, in some embodiments, the multicellular structure includes a wheat, rice or barley plant or a part, organ or tissue thereof, as hereinbefore described.

In some embodiments, the nucleotide sequence of interest includes a nucleotide sequence which, when expressed by one or more cells of a plant, improves the stress tolerance of the plant as hereinbefore described.

In some embodiments, the nucleotide sequence of interest encodes a DREB polypeptide as hereinbefore described. In some embodiments, the DREB polypeptide is a TaDREB3-like polypeptide as hereinbefore described.

In some embodiments, the present invention also provides a plant or a part, organ or tissue thereof having improved stress tolerance, wherein the plant comprises one or more cells of the third aspect of the invention.

In some embodiments of the fourth aspect of the present invention, the plant or a part, organ or tissue thereof has improved stress tolerance relative to a plant or a part, organ or tissue thereof which does not include one or more cells of the third aspect of the invention.

A plant or a part, organ or tissue thereof according to the fourth aspect of the invention may be regenerated from transformed plant material such as transformed callus, cultured embryos, explants or the like using standard techniques of the art. Such plants are typically referred to as T₀ plants. Plants according to the third aspect of the invention should also be understood to include progeny of T₀ plants. Such progeny plants may result from self fertilisation of the T₀ plants or crossing of the T₀ plants with one or more other plants of the same species, or of a different species to form hybrids. As will be appreciated, the construct of the second aspect of the invention may segregate in progeny plants, and thus the plants of the fourth aspect of the invention extend only to those progeny plants that include the construct.

As used herein, the singular forms “a”, “an” and “the” include plural aspects unless the context already dictates otherwise.

Where a range of values is expressed, it will be clearly understood that this range encompasses the upper and lower limits of the range, and all values in between these limits.

“About” as used in the specification means approximately or nearly and in the context of a numerical value or range set forth herein means±10% of the numerical value or range recited or claimed.

Finally, reference is made to standard textbooks of molecular biology that contain methods for carrying out basic techniques encompassed by the present invention, including DNA restriction and ligation for the generation of the various genetic constructs described herein. See, for example, Sambrook and Russell, Molecular Cloning: A Laboratory Manual (3rd edition). Cold Spring Harbor Laboratory Press, 2001.

The present invention is further described by the following non-limiting examples. It is to be understood that the following description is for the purpose of describing particular embodiments only and is not intended to be limiting with respect to the above description.

### Example 1

### Transgene Analysis in Wheat

Analysis of wheat plants containing a WRKY71 promoter transgene was conducted as set out below.

### Gene Cloning and Plasmid Construction

Genomic DNA isolated from Oryza sativa L. ssp. Japonica cv. Nipponbare was used as a template for cloning of the WRKY71 promoter. In this regard, a 2331 bp long fragment from the sequence upstream of the translation start codon of OsWRKY71 was amplified by PCR using primers with introduced HindIII and KpnI restriction sites. The sequence of the PCR amplified WRKY71 promoter is set forth in SEQ ID NO: 1. The primer sequences used for PCR were 5′-GCCAAGCTTCTTAGTAAACGACCCAAC-3′ (SEQ ID NO: 2—OsWRKY71F) and 5′-CCCGGTACCCGGCGAACGATTTATCAC-3′ (SEQ ID NO: 3—OsWRKY71R), respectively.

The PCR-amplified promoter fragment was isolated and cloned into the HindIII-KpnI restriction sites of the pMDC32 vector, as briefly described below. The pMDC32 vector was linearised by simultaneous restriction with HindIII and KpnI and purified from a 2% agarose gel using a Gel extraction Kit (Scientifix). Linearisation of the vector led to excision of the 2×35S promoter of the vector. The PCR-amplified WRKY71 promoter fragment was purified with a PCR clean-up Kit (Scientifix), and was then digested with the restriction enzymes HindIII and KpnI. The digested product was purified from an agarose gel and ligated into the linearized pMDC32 vector using T4 ligase (Invitrogen). The ligation mix was transformed into competent DB3.1 E. coli cells (Invitrogen) and plated on Kan/Cm agar plates. Plasmid DNA was purified from transformed colonies using the ISOLATE DNA Kit (BIOLINE). The generated binary vector, designated pWRKY71, was verified by sequencing.

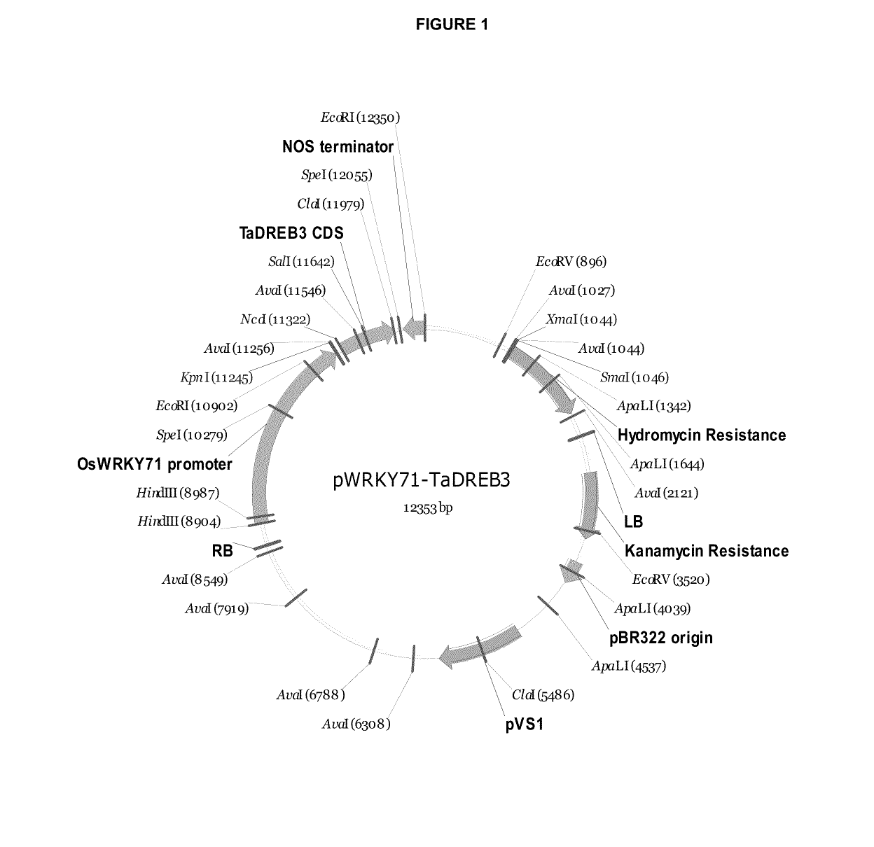

The pWRKY71 vector was used to clone the TaDREB3 coding sequence downstream of the WRKY71 promoter by recombination with EcoRV linearised pENTR-D-TOPO-TaDREB3 plasmid using Gateway LR Clonase II Enzyme Mix (Invitrogen). The resulting transgene construct, the structure of which is shown in FIG. 1, was designated pWRKY71-TaDREB3. Selectable marker genes present in the construct conferred hygromycin resistance in plants and kanamycin resistance in bacteria.

### Plant Transformation

The pWRKY71-TaDREB3 transgene construct was transformed into wheat (Triticum aestivum L. cv. Gladius) using biolistic bombardment as described by Kovalchuk N et al., 2009 (supra). Twenty five independent transgenic wheat lines were generated.

### Analysis of Transgene Expression

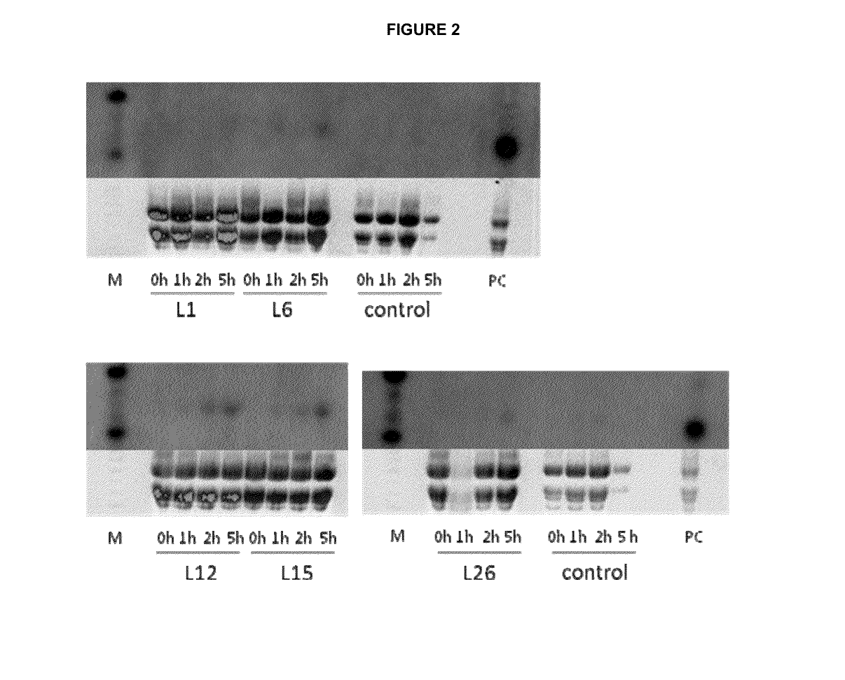

Seeds from the transgenic wheat lines were germinated for 4 days on moist filter paper before being transferred to a supported hydroponic setup. Seedlings were transplanted into individual 280 mm long×40 mm diameter tubes filled with 3 mm diameter polycarbonate fragments, used as a soil substitute. Plants were provided with a growth solution containing 5 mM KNO₃, 2 mM Ca(NO₃)₂, 2 mM MgSO₄, 0.5 mM Na₂SO₃, 0.2 mM NH₄NO₃, 0.1 mM KH₂PO₄, 0.05 mM NaFe(III)EDTA and micronutrients (50 μM H₃BO₃, 10 μM ZnSO₄, 5 μM MnCl₂, 0.5 μM CuSO₄ and 0.1 μM Na₂MoO₄), pH=6.5-7.0, in a 20 min pump/20 min drain cycle, for approximately 10 days, until the third leaf emerged. Seedlings were incubated at 4° C. for 1, 2, 3 and 5 hours. Twelve T₁ plants were grown for each of 7 independent transgenic wheat lines. Shoots and roots were collected from 3 plants for each time point and analysed using northern blot hybridisation (FIG. 2). Transgenic lines L6 and L26 showed (visible) induction of expression of the transgene at the 5 hour time point, line L15 showed (visible) induction of expression after 2 hours, while line L12 showed (visible) induction of expression after 1 hour.

Due to the moderate strength of the promoter, results of northern blot hybridisations were weak. In order to further explore the expression induction of the transgene, a quantitative PCR approach, according to the protocol described by Burton R A et al., 2004 (Plant Physiol. 134: 224-236), was used to investigate expression of transgene (TxDREB3), endogene (TaDREB3) and a potential target gene (TaCor14B) in three selected transgenic lines, L6, L12 and L15. The potential target gene, Cor14B, is a wheat gene previously shown to be expressed in response to cold (Tsvetanov S et al., 2000, Genes Genet. Syst. 75: 49-57).

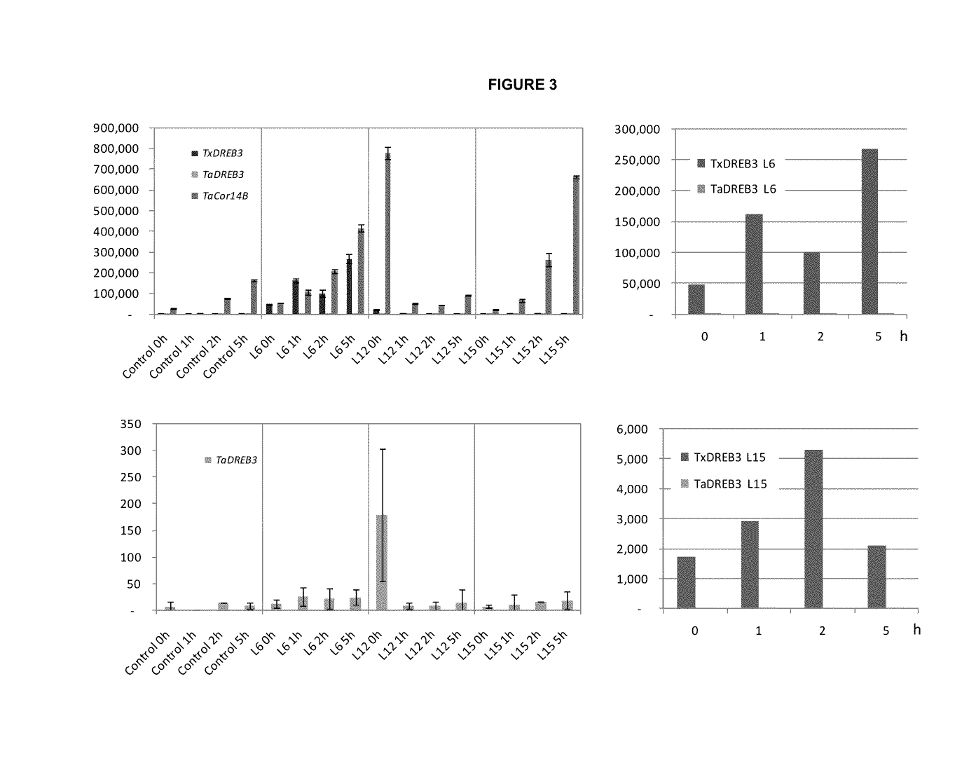

As can be seen in FIG. 3, two of the three analysed transgenic lines (L15 and L6) demonstrated different levels of transgene expression (i.e. PR0189 promoter activation) as a result of cold. The level of transgene (TxDREB3) activation in these transgenic lines was 500 to 200,000 fold higher, respectively, than levels of expression of the endogene (TaDREB3). Transgene expression occurred immediately in response to cold (1-hour time point) in both lines, with maximum expression being seen after 2 hours at 4° C. for line L15, and after 5 hours at 4° C. for line L6. A basal level of expression from the WRKY71 promoter was observed in each line (time-point 0). Induction of the potential target gene, TaCor14B, was 2-3 fold higher in transgenic lines than in control wheat plants. Maximum expression of the potential target gene in all lines could be seen after 5 hours at 4° C.

These results show that under the presence of stress (in this instance cold), expression from the WRKY71 promoter is increased.

### Analysis of Frost Tolerance

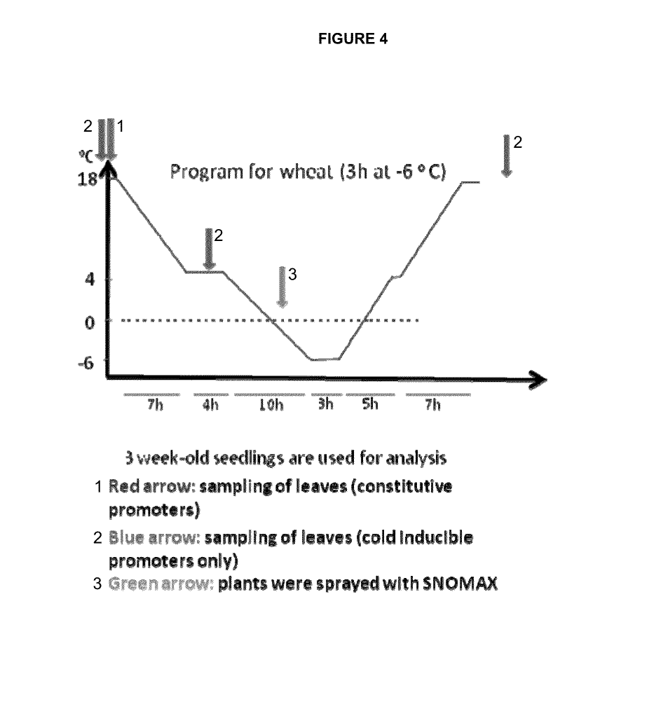

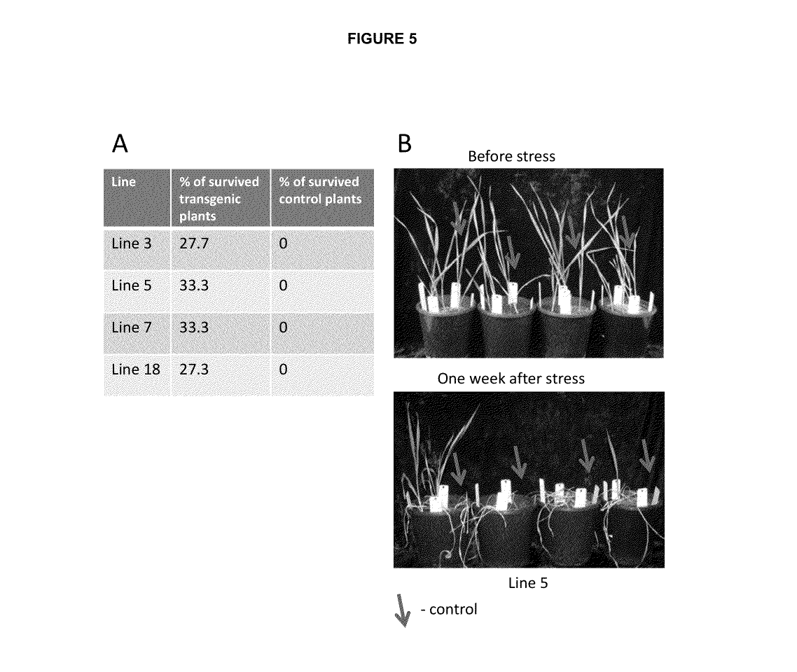

A frost tolerance test, with minimum temperature −6° C. for 3 h, was performed in a cold/frost cabinet on 3-week-old seedlings of various T₁ transgenic and control wheat plants. FIG. 4 shows the incubation protocol used for the frost tolerance test. Under the protocol conditions, all plants were severely damaged; however, none of the control plants were able to recover after 2 weeks at normal temperatures. Under the same conditions, 4 transgenic lines demonstrated increased survival as shown in the Table in FIG. 5. A representative transgenic line (L5) is depicted prior to, and one week after, stress induction. No differences in development of the control plants or transgenic plants before stress were detected. No differences were detected between control and transgenic plants, which survived frost, after several weeks of recovery.

### Example 2

### Transgene Analysis in Barley and Rice

Analysis of barley and rice plants containing a OsWRKY71 promoter transgene or a TdCor39 promoter transgene was conducted as set out below.

### Methods

### Gene Cloning and Plasmid Construction

The pWRKY71-TaDREB3 transgene described above in Example 1 was used for the experiments conducted in this Example.

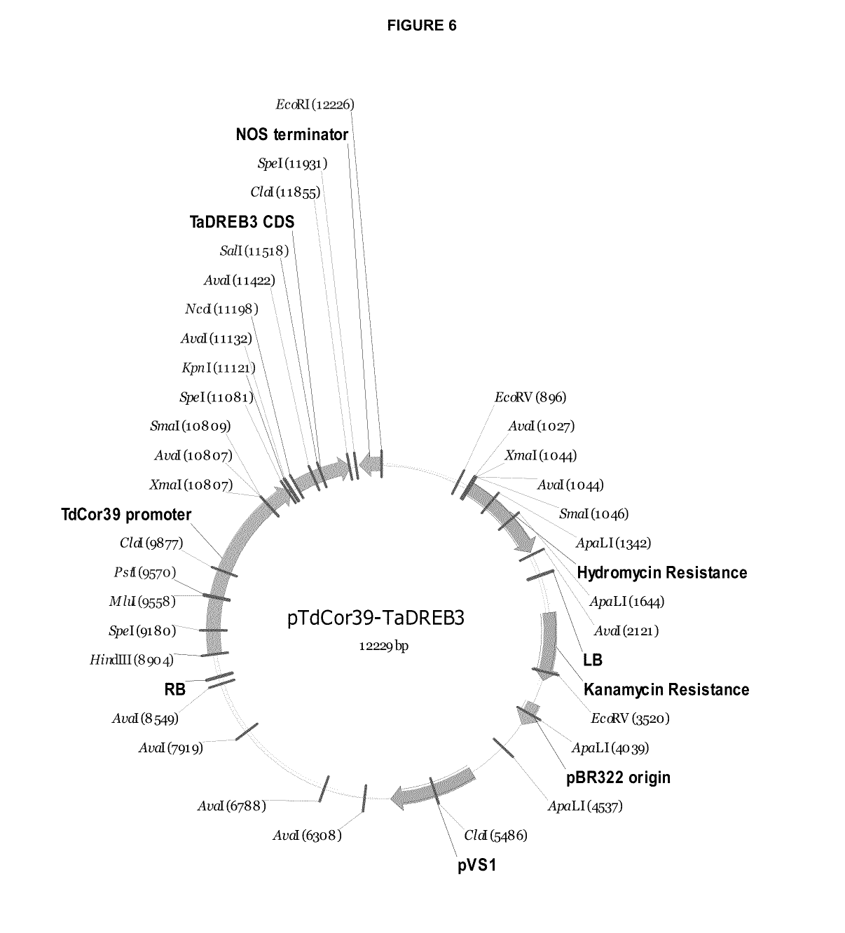

With respect to Cor39, the full-length coding region of the TaCor39 cDNA (GenBank Accession Number AF058794) was amplified by PCR using a cDNA library obtained from spikes of drought-stressed wheat (Triticum aestivum L cv. Chinese spring) as a template. The TaCor39 cDNA was used as a probe to screen a BAC library prepared from genomic DNA of Triticum durum cv. Langdon (Cenci A et al., 2003, Theoretical and Applied Genetics 107: 931-939), as described by Kovalchuk N et al., 2009 (supra). The T. durum homolog of the TaCor39 gene was amplified by PCR using DNA of the selected BAC clone (#891 H17) as a template and primers derived from the coding region of TaCor39 cDNA. The primer sequences used for the PCR were 5′-ATGGAGAACCAGGCACACATC-3′ (SEQ ID NO: 4—TaCor39F) and 5′-GGTCATTCCAGTGTGTGCAT-3′ (SEQ ID NO: 5—TaCor39R). The gene of the T. durum orthologue of TaCor39 was designated TdCor39. The protein product of TdCor39 had no differences in amino acid sequence from TaCor39. The TdCor39 promoter sequence was identified through the sequencing of the BAC clone as described by Kovalchuk N et al., 2009 (supra). A 2207 bp long fragment from the sequence upstream of the translation start codon of TdCOR39 was amplified by PCR. The sequence of the PCR amplified Cor39 promoter is set forth in SEQ ID NO: 6. The primer sequences used for PCR were 5′-CACCTGTTACAAGATAGCATC-3′ (SEQ ID NO: 7—TdCor39F) and 5′-CTTGCGCTGAGCTTCTGACTC-3′ (SEQ ID NO: 8—TdCor39R), respectively. The PCR product was used to generate a pCor39 vector, and subsequently a pCor39-TaDREB3 transgene using the methods as described above in Example 1 for the WRKY71 promoter. The structure of the pCor39-TaDREB3 transgene construct is shown in FIG. 6.

### Plant Transformation and Analysis of Transgenic Plants Under Abiotic Stress

The pCor39-TaDREB3 and pWRKY71-TaDREB3 constructs were transformed into barley (Hordeum vulgare L. cv. Golden Promise) and rice (Oryza sativa L. ssp. japonica cv. Nipponbare), using Agrobacterium-mediated transformation (Matthews P R et al., 2001, Molecular Breeding 7: 195-202; Tingay S et al., 1997, Plant Journal 11: 1369-1376). Transgene integration was confirmed by PCR using the forward primer from the 3′ end of the promoter (5′-GTATCTCGCATATGGACGGAG-3′—SEQ ID NO: 9—PF3′) and the reverse primer from the 5′ end of the nos terminator (5′-TTGCCAAATGTTTGAACGATC-3′—SEQ ID NO: 10—NTR5′). The transgene copy number was estimated in T₁ progeny of selected transgenic lines using quantitative PCR (Q-PCR). Briefly, transgene copy number was estimated by efficiency adjusted real-time Q-PCR. A modified ΔΔCt method adjusted for amplification efficiency was used to determine the number of copies of the transgene per genome in each sample (Yuan J S et al., 2008, Biotechnol. J. 2008: 112-123). DNA was extracted from leaf tissue using a method described by Shavrukov Y et al., 2010 (Genomics 10, 277-291). Prior to use in real-time Q-PCR, each DNA sample was diluted with sterile deionised water to be within the copy-standard serial dilution range (12.5 ng/μl to 200 ng/μl). For template loading normalisation, PCRs were performed using primers and probes complimentary to single-copy endogenous reference genes. In the case of Hordeum vulgare (barley) and Triticum aestivum (wheat), primers and a TaqMan probe complimentary to either a portion of the Hordoindoline-b (Hin-b) gene, which is an orthologue of the wheat Puroindoline-b (Pin-b) gene, or to the Pin-b gene (Li Z et al., 2004, Plant Mol. Biol. Rep. 22, 179-188), were used. The sequences of the primers and probe used were 5′-ATTTTCCAGTCACCTGGCCC-3′ (SEQ ID NO: 11—HPF) and 5′-TGCTATCTGGCTCAGCTGC-3′ (SEQ ID NO: 12—HPR), and dual-labelled TaqMan probe 5′-CAL fluor Gold 540-ATGGTGGAAGGGCGGCTGTGA-BHQ1-3′ (SEQ ID NO: 13—TMBW). For Oryza sativa (rice), primers and a TaqMan probe specific to the sucrose phosphate synthase gene were used (Ding J et al., 2004, Biotechnology 52: 3372-3377). The sequences of the primers and probe used were 5′-TTGCGCCTGAACGGATAT-3′ (SEQ ID NO: 14—SPSF) and 5′-CGGTTGATCTTTTCGGGATG-3′ (SEQ ID NO: 15—SPSR), and dual-labelled TaqMan probe 5′-FAM-GACGCACGGACGGCTCGGA-BHQ1-3′ (SEQ ID NO: 16—TMR). For the various transgenes analysed, a portion of the hygromycin resistance gene (Hyg) was used as a target sequence. The sequences of the primers and TaqMan probe used were 5′-CGCTCGTCTGGCTAAGATCG-3′ (SEQ ID NO: 17—HygF) and 5′-AGGGTGTCACGTTGCAAGAC-3′ (SEQ ID NO: 18—HygR), and dual-labelled TaqMan probe 5′-FAM-TGCCTGAAACCGAACTGCCCGCTG-BHQ1-3′ (SEQ ID NO: 19—TMTr). Real-time Q-PCRs were performed on a LightCycler 480 thermal cycler. Each PCR was comprised of 1×IQ Supermix (Bio-Rad); forward and reverse primers (400 nM each); dual-labelled probe (200 nM); DNA (2 μl) and deionised water to a total volume of 10 μl. The thermal cycling parameters were 95° C. for three minutes followed by 40 cycles of 95° C. for 15 seconds and 60° C. for 60 seconds with fluorescence readings acquired at each cycle on the yellow and green channels.

To allow for the calculation of transgene copy number from unknown DNA samples, a copy-standard serial dilution series was set up. Genomic DNA from a plant known to contain a single copy of the hygromycin gene in addition to the single-copy endogenous reference gene was extracted and diluted. Amounts of 400 ng, 200 ng, 100 ng, 50 ng and 25 ng were used. Three replicate PCRs for each unknown sample and each diluted copy-standard sample were performed with each primer/probe set. Ct values were calculated using the supplied software for the LightCycler 480 thermal cycler. The PCR efficiency for each primer/probe set was determined via analysis of the Ct values obtained from the diluted copy-standard series. Subsequently, all Ct values were adjusted using these PCR efficiency values prior to calculating ΔCt_{adjusted} (ΔCt_{adjusted}=Hyg Ct_{adjusted}−reference gene Ct_{adjusted}). The transgene copy number for each unknown sample was determined by calculating 2^{-ΔΔCt}_{adjusted}. (ΔΔCt_{adjusted} is defined as the difference between the average ΔCt_{adjusted} of the copy standard series and the ΔCt_{adjusted} of the unknown sample). Calculated transgene copy numbers were rounded to the closest integer.

The basal level of activity of the TdCor39 and OsWRKY71 promoters in unstressed leaves of transgenic T₀ lines was demonstrated by northern blot hybridization analysis.

Transgenic barley plants (Hordeum vulgare cv Golden Promise) were grown in either a growth room (for cold and drought tests) or in a glasshouse (for characterisation of plant phenotypes). Growth room temperatures were maintained at 24° C. during the 12 daylight hours and 18° C. during the night, and the average relative humidity was 50% during the day and 80% during the night. Wild type plants were used as a control. For experiments hydroponics seeds were initially germinated at room temperature in Petri dishes on wet filter paper. After two or three days, germinated seeds were transferred to hydroponic boxes immersed into growth solution (Johnson A A et al., 2011, PLoS One 6(9):e24476. Epub 2011 Sep. 6). Two-week old seedlings were either subjected to cold stress at 4° C. or to dehydration by withholding growth solution for several hours. For quick analyses of promoter induction by dehydration the leaf tissues of two-week old seedlings grown on hydroponics were detached and dried at room temperature for 7 hours. After this treatment, leaves were stored at −80° C. until RNA isolation. In the case where experiments were performed in pots, seeds were germinated directly in soil. For cold treatment, control and transgenic seedlings were grown first for three weeks in soil in a growth room and were then transferred to a cold cabinet (BINDER GmbH, Tuttlingen, Germany) and kept at constant temperatures of either 2° C. or 4° C. for up to 8 hours. Leaves were collected for RNA isolation before stress was applied and during cold treatment at 0, 2, 5 and 8 hours.

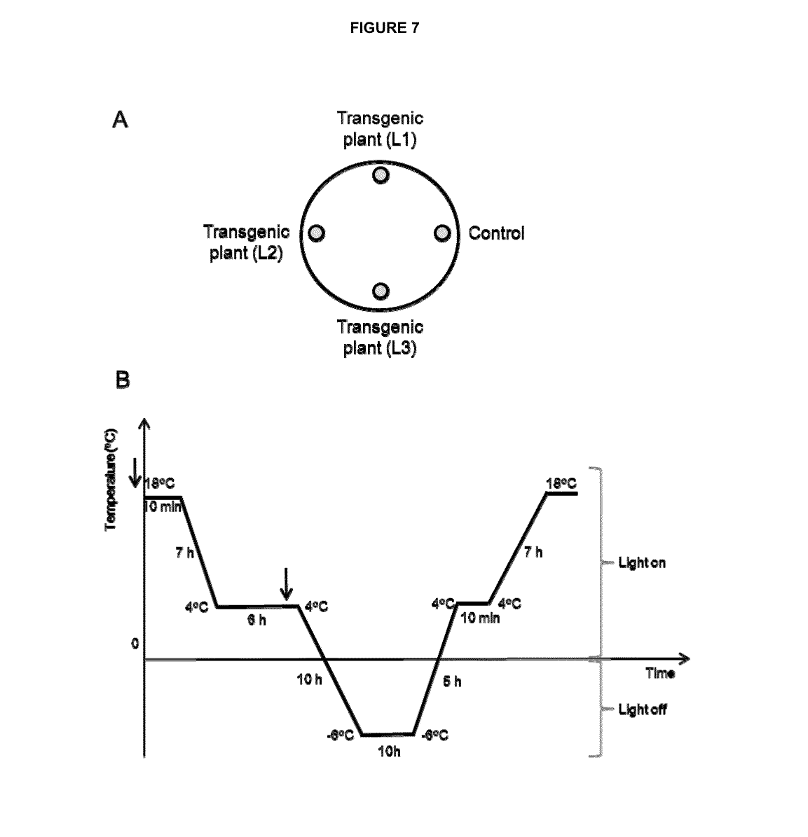

For frost survival tests, three-week old seedlings were exposed to gradual temperature decreases and then increases with a maximum of 18° C. and a minimum of −6° C. (FIG. 7). Leaf tissue was collected before stress and after the temperature had decreased to 4° C. (FIG. 7). When the temperature reached 0° C. plants were sprayed with a 2 g/L solution of Snomax (York Snow, Victor, N.Y.) to initiate simultaneous ice crystallization before the temperature reached −6° C. The temperature then decreased to minus 6° C. for 10 hours and treatment continued according to the regime described in FIG. 7. When the temperature returned to 18° C., plants were transferred to the growth room to recover. The number of surviving plants was estimated after two weeks of recovery; plants which survived were re-potted and transferred to a glasshouse for seeds.

Transgenic rice plants (Oryza sativa L. ssp. japonica cv. Nipponbare) were grown in the growth cabinet. Growth cabinet temperatures were maintained at 24° C. during the 12 hours incubation with light and at 18° C. during the night time; the average relative humidity was 50% during the day and 80% during the night. Before sowing the rice seeds they were incubated in 70% ethanol for 1 minute. Ethanol was then replaced with 30% bleach solution (Domestos, NSW, Australia) containing a drop of TWEEN20, for 30 min. The rice seeds were rinsed 3 to 5 times with MiliQ water and then incubated with shaking in one more portion of MiliQ water for 10 minutes. Seven to ten seeds were placed onto Petri dishes with rice growth media (1% of potato dextrose powder in MiliQ water) and were grown at 28° C. and 12 hours dark/light period. After one week, some germinated seeds were transferred to either small pots filled with soil or to hydroponic boxes. The rice seedlings were grown in the hydroponic boxes and/or pots for two weeks and were then subjected to cold. Three-week old seedlings which were grown in a hydroponic box were incubated in a cold chamber at a constant temperature of 4° C.

### RNA Isolation and Northern Blot Hybridisation

Collected plant material was immediately frozen in liquid nitrogen and stored at −80° C. until RNA extraction. Total RNA was isolated from wild type and transgenic plant leaf tissue using the TRIzol kit (Invitrogen, Victoria, Australia). RNA was electrophoretically separated on a 1.3% agarose gel containing 6% formaldehyde, transferred to nylon membrane and hybridized with ³²P-labeled DNA probes according to the protocol described by Church G M and Gilbert W, 1984 (Proc. Natl. Acad. Sci. USA 81: 1991-1995).

### Quantitative PCR (Q-PCR)

Q-PCR analyses of expression of the Cor39 gene in different tissues and under several stresses were performed as described by Burton R A et al., 2008 (Plant Physiology 146: 1821-1833). For gene expression analysis the cDNA tissue series were prepared from different tissues of T. aestivum cv. Chinese spring as described by Morran S et al., 2011 (Plant Biotechnol. J. 9: 230-249). The stress cDNA series for Q-PCR analysis were prepared from three to four leaves from each of 2-4 six week-old plants of either T. aestivum cv. RAC875 or T. durum cv. Langdon subjected to each of the following stresses: cold stress at 4° C. (samples were collected 0, 1, 4, 24, and 48 hours after stress application), drought (samples were collected from well watered plants growing in soil, and plants at different stages of drought until plants became strongly wilted (VWC in soil was 3%)), wounding with a metal brush (samples of T. durum were collected at 0, 0.25, 0.5, 1, 1.5, 2, 3, 4, 7, 12, 24, 36, and 48 hours after wounding). Analysis of transgene expression and expression of downstream genes was analysed in transgenic lines selected by Northern blot analysis. The specific primers used in the Q-PCR are listed in Table 2.

TABLE 2

| | Description | | |
|---|---|---|---|
| | and | | |
| Gene | purpose | Forward primer sequence | Reverse primer sequence |
| TaDREB3 | Endogene | CTCGATTCGCTTGCTCCTCAG | TCCTGATGACAAGCTGTAGTGTGC |
| | Q-PCR | (SEQ ID NO: 20 - TaDREB3F) | (SEQ ID NO: 21 - TaDREB3R) |
| TxDREB3 | Transgene | GTATCTCGCATATGGACGGAG | TTGCCAAATGTTTGAACGATC |
| | Q-PCR, | (SEQ ID NO: 22 - TxDREB3F) | (SEQ ID NO: 23 - TxDREB3F) |
| | selection of | | |
| | transgenic | | |
| | lines | | |
| HvCor14B | Cold- | TTGAGGATGTGAGCAAATGAG | TACATCGTCAATGACGAGACC |
| | responsive | (SEQ ID NO: 24 - HvCor14BF) | (SEQ ID NO: 25 - HvCor14BR) |
| | Q-PCR | | |
| HvDhn8 | Cold- | GCGAGCACAAGCCCAGAG | GCCCAGCAATAAACCAATACACA |
| | responsive | (SEQ ID NO: 26 - HvDhn8F) | (SEQ ID NO: 27 - HvDhn8R) |
| | Q-PCR | | |
| HvDhn5 | Cold- | TGGCGAAGTTCCACCGTATGC | ACGAAAACTGTTGCCACACTG |
| | responsive | (SEQ ID NO: 28 - HvDhn5F) | (SEQ ID NO: 29 - HvDhn5R) |
| | Q-PCR | | |
| HvA22 | Cold- | GCTCCTCACCCACCTCCACTCC | CTGAGCTGCTCCCTGACGACCTT |
| | responsive | (SEQ ID NO: 30 - HvA22F) | (SEQ ID NO: 31 - HvA22R) |
| | Q-PCR | | |

### Results

### Analysis of TdCor39 and OsWRKY71 Gene Expression and Cloning of Promoters

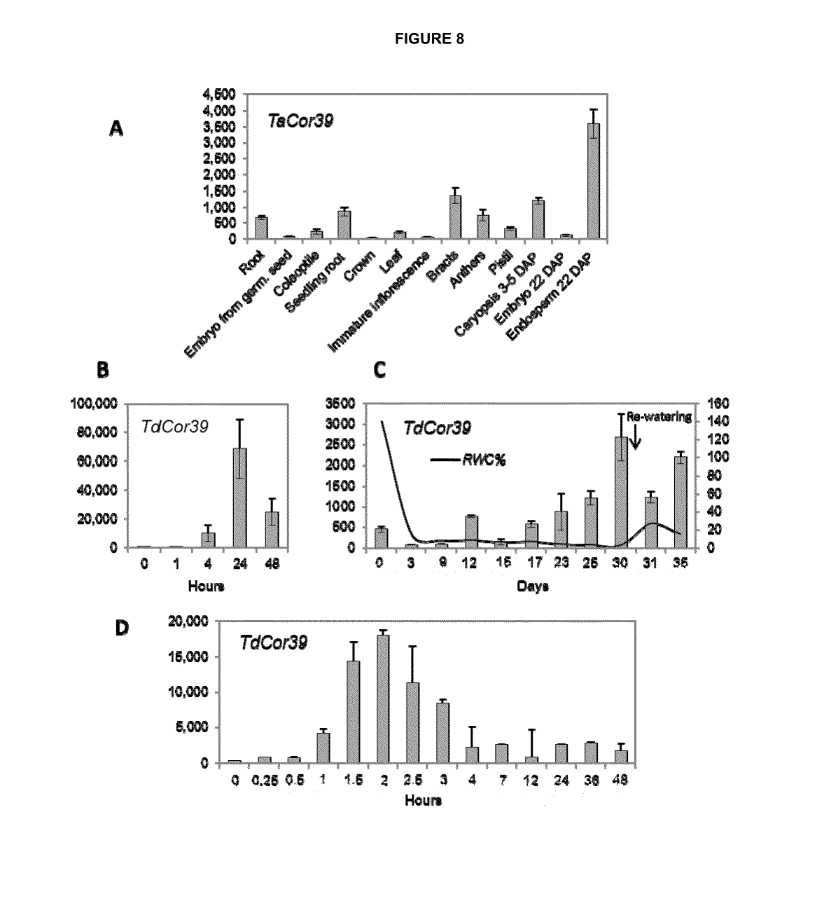

Expression of the Cor39 gene was analysed in different tissues of unstressed bread wheat plants. Low levels of expression were detected in embryos, leaves and coleoptiles, and higher levels were seen in roots and reproductive tissues. The highest level of expression was observed in mature endosperm (FIG. 8A). In leaves of durum wheat the Cor39 gene was strongly activated by cold and moderately by drought (FIG. 8 B,C). Induction by cold was initially detected after 4 hours of incubation at 4° C. and reached a maximum after several hours. After 48 hours of cold treatment the level of TdCor39 expression remained high (FIG. 8B). In contrast, activation by wounding was more moderate than by cold, but it was detected much earlier (15 minutes after stress was applied). Expression of the TdCor39 gene reached its maximum two hours after wounding. Four hours after stress it returned to a low level and remained at this level for at least 48 hours (FIG. 8D).

### Comparison of Developmental Phenotypes of Transgenic Barley Plants

Seventeen independent transgenic barley lines transformed with the pCOR39-TaDREB3 construct, and fourteen transgenic barley lines transformed with the pWRKY71-TaDREB3 construct, were selected by PCR using transgene-specific primers. Five independent transgenic lines (L5, L12, L18, L19, and L20) transformed with the pCOR39-TaDREB3 construct and three lines (L2, L5, and L16) for the pWRKY71-TaDREB3 construct were selected for further analysis. The selection was based on basal levels of transgene expression in T₀ plants, and/or on results of promoter activation by cold and dehydration in T₁ progeny obtained in the hydroponics (data not shown).

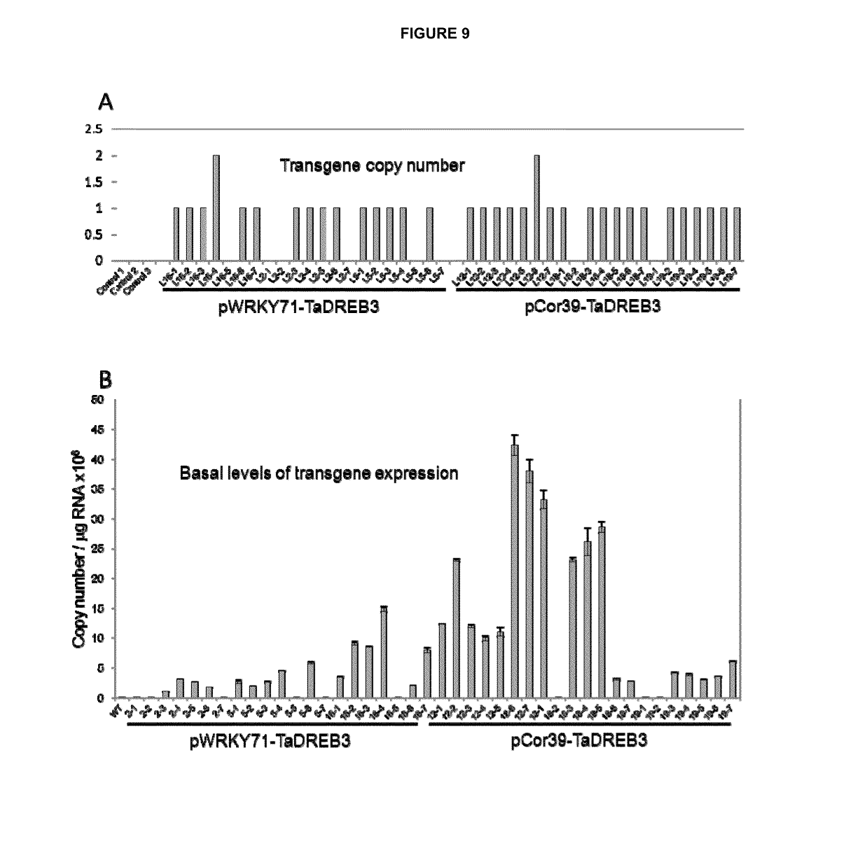

Six of selected transgenic lines (three for each promoter) were used for the comparison of phenotypes and grain yields of transgenic and control plants. Seven T₁ plants for each transgenic line and seven control plants (wild type barley) were grown in the glasshouse and transgene copy number and basal levels of transgene expression were examined for each plant (FIG. 9). All T₁ plants except L16-4 (WRKY71 promoter) and L12-6 (Cor39 promoter) contained a single copy of the transgene (FIG. 9A). All plants of Line 12 and four plants of Line 18 had high basal levels of transgene expression (FIG. 9B). Eight null segregants (N) were detected by Q-PCR, and were combined in a second group of control plants for phenotypic analysis.

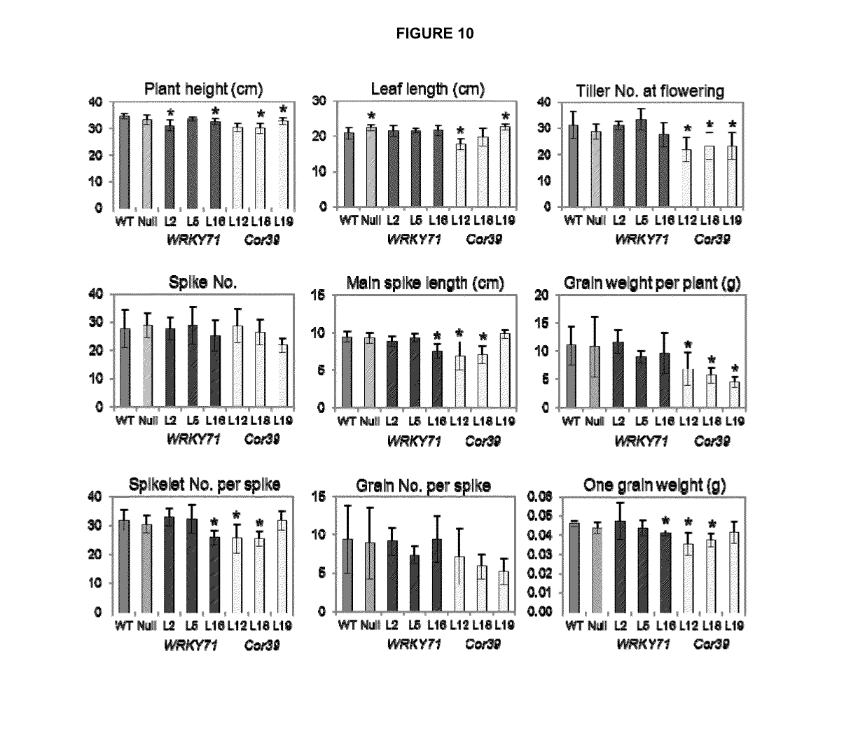

Results of the phenotypic analysis are shown in FIG. 10. Plant height and the length of leaves were measured at the end of the fourth week after germination. Tiller number was evaluated at the beginning of flowering. Spike number, spike length, number of spikelets per spike, grain number per spike and grain weight were analysed after harvest. As seen in FIG. 10, phenotypic analysis of transgenic plants revealed a small decrease in plant height for both types of transgenic plants when compared with control plants. No difference in leaf length was observed for plants transformed with pWRKY71-TaDREB3 construct. However, significant variability of leaf length was found for pCor39-TaDREB3 transgenic barley lines. The number of tillers at flowering was significantly smaller in transgenic barley transformed with pCor39-TaDREB3 construct, although the number of harvested spikes was roughly the same as for control plants. In contrast, application of the OsWRKY71 promoter resulted in no differences in the number of tillers or spikes between transgenic and control plants. However, the length of the spikes and the number of spikelets per spike were significantly smaller in one of the WRKY71 lines (L16) and two of the Cor39 lines (L12 and L18). In Line 16 (WRKY71 promoter) this correlated with a much stronger level of transgene expression than in the other two tested lines. The total grain weight per plant, grain number per spike and weight of one grain were lower in transgenic barley transformed with the pCor39-TaDREB3 construct than for control plants. In contrast, grain yield from barley transformed with pWRKY71-TaDREB3 was the same or close (L16) as compared to control plants.

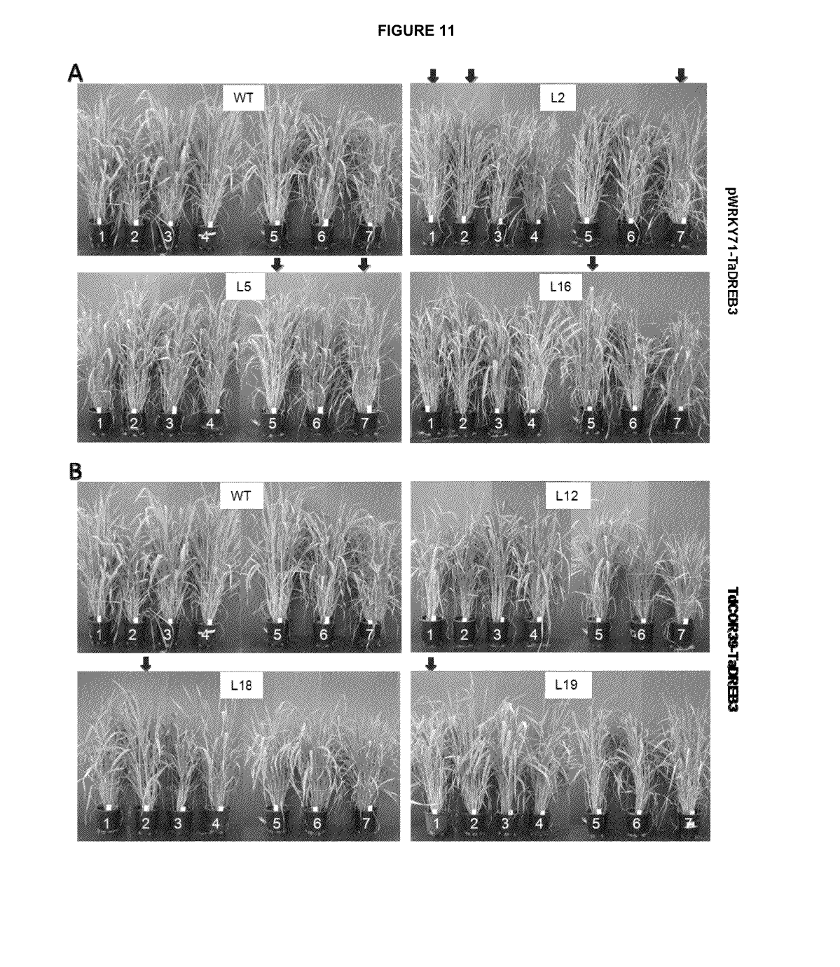

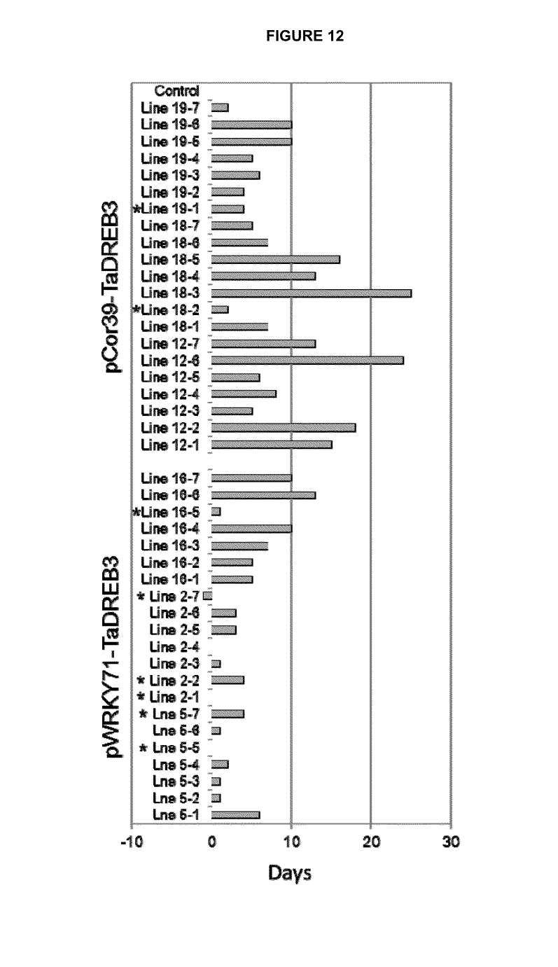

Pictures of all analysed transgenic and control plants two weeks before flowering are depicted in FIG. 11. As shown in FIG. 12, both types of transgenic plants had a delay in flowering. Average delays in flowering time of transgenic barley plants versus control plants were 9.8 days for the TdCor39 promoter and 3.6 days for the OsWRKY71 promoter. Importantly, these delays were much shorter than the 3-6 weeks delay in flowering of barley T₁ plants transformed with 2×35S-TaDREB3 construct (Morran et al. 2011, supra). Interestingly, some null segregants had up to 4 days delay in flowering time.

### Frost Tolerance Tests of Transgenic Barley Plants

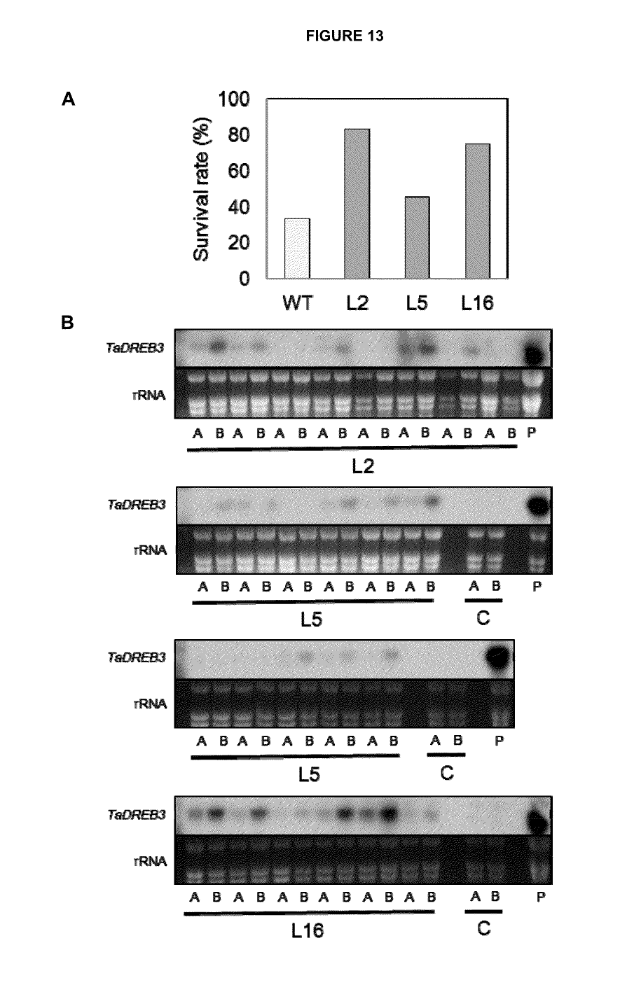

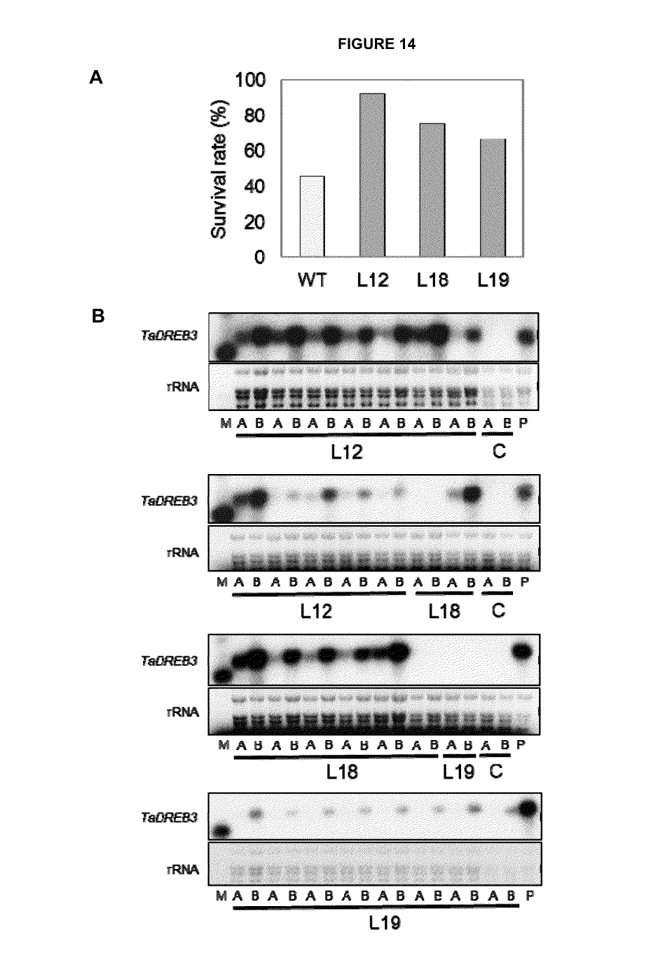

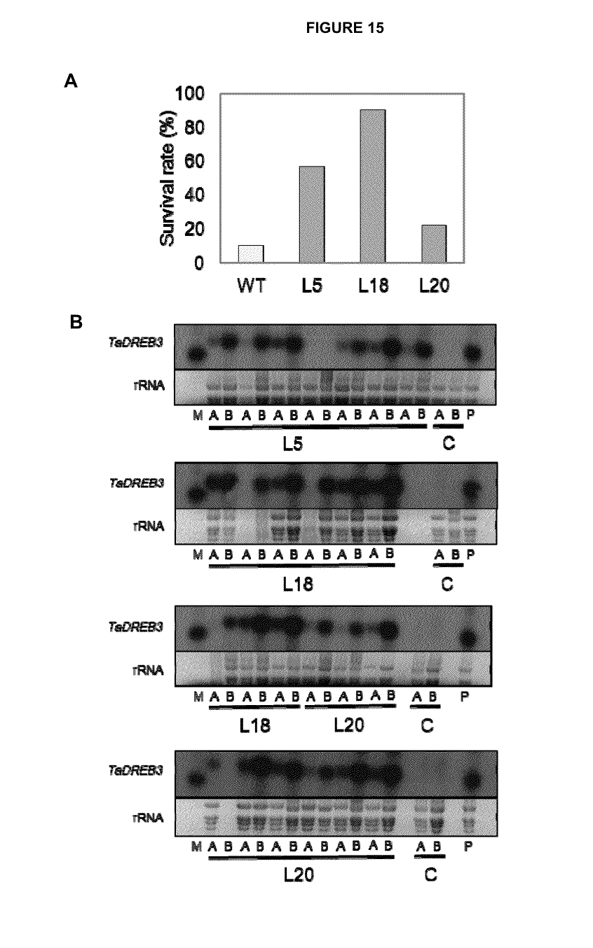

Four week-old T₁ barley seedlings, 6-12 plants for each transgenic line, were used for frost survival tests in an automatic cold cabinet. Design and conditions of the experiment have been described by Morran et al., 2011 (supra) and can be seen in FIG. 7. Three transgenic lines (L2, L5, and L16) for the pWRKY71-TaDREB3 construct, and five transgenic lines (L5, L12, L18, L19, and L20) for the pCor39-TaDREB3 construct were tested in three independent experiments. The results of this analysis are shown in FIGS. 13, 14 and 15, respectively. All transgenic lines demonstrated significantly higher survival rates than control (wild type) plants. Transgene expression before the cold treatment and after 7 hours of temperature decrease from 18 to 4° C. and following 5 hour long incubation at 4° C. was assessed by Northern blot hybrydization. Most of the transgenic plants demonstrated strong transgene activation by cold before the below 0° C. incubation had commenced; some plants had relatively high basal levels of transgene expression, although the results of the Northern blot hybridization can only be considered as semi-quantitative. Plants where transgene expression was not detected were excluded from the survival rate data.

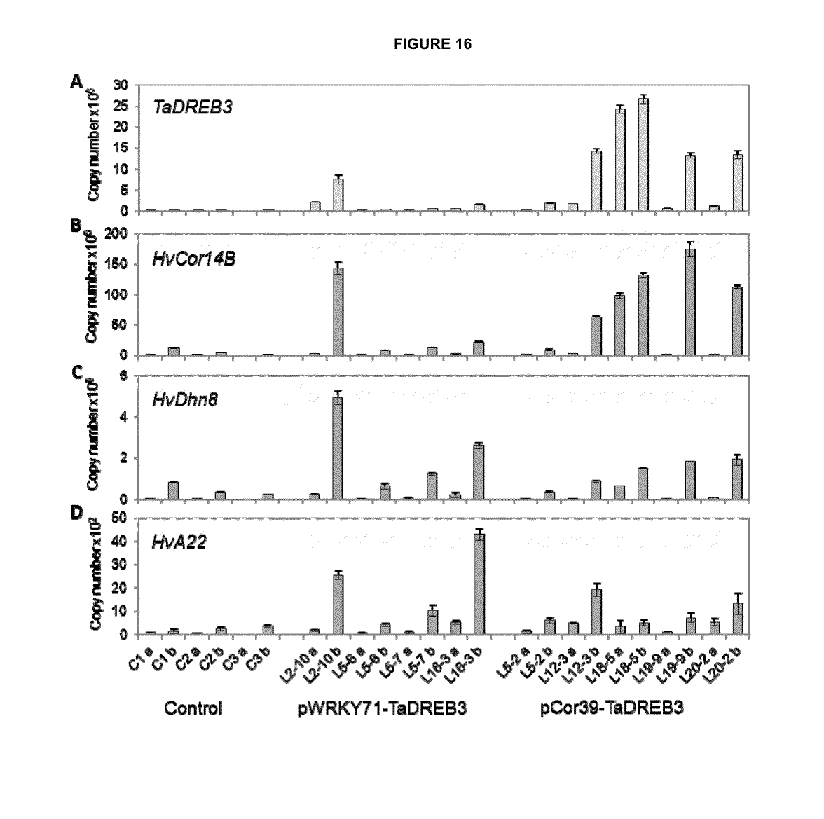

Three T₁ plants for each construct (one plant per transgenic line) were selected for the analysis of transgene expression. As shown in FIG. 16A, all plants except L18-5 demonstrated a low to moderate basal level of activity and strong activation of the TaDREB3 transgene by cold. The 18-5 line had a very high basal level of promoter activity and a very small further activation of the TdCor39 promoter by cold. Overall expression was stronger in plants transformed with the pCor39-TaDREB3 construct. Expression of three cold responsive genes, HvCor14b, HvDhn5 and HvA22, were tested in the same transgenic plants with the aim to compare levels of expression of the transgene and downstream target genes thus indirectly confirming the presence of the functional TaDREB3 protein. As shown in FIGS. 16B, C and D, respectively, all three genes were induced by cold in control plants. However, their induction by cold in transgenic plants was much stronger. As previously observed in barley plants with constitutive expression of TaDREB3 (Morran et al. 2011, supra), expression levels of the HvCor14b gene correlated well with levels of transgene expression, suggesting possible direct regulation of this gene by TaDREB3 (FIG. 16B). There was less correlation between the expression patterns of the transgene and the two other tested genes, HvDhn5 and HvA22, although the expression levels of both of the genes in all transgenic plants were higher than in control plants. Surprisingly, transgenic plants transformed with the pWRKY71-TaDREB3 construct showed an overall lower up-regulation of the transgene at the transcriptional level than the plants transformed with the pCor39-TaDREB3 construct but stronger up-regulation of downstream genes.

### Spatial Activity of TdCor39 and OsWRKY71 Promoters

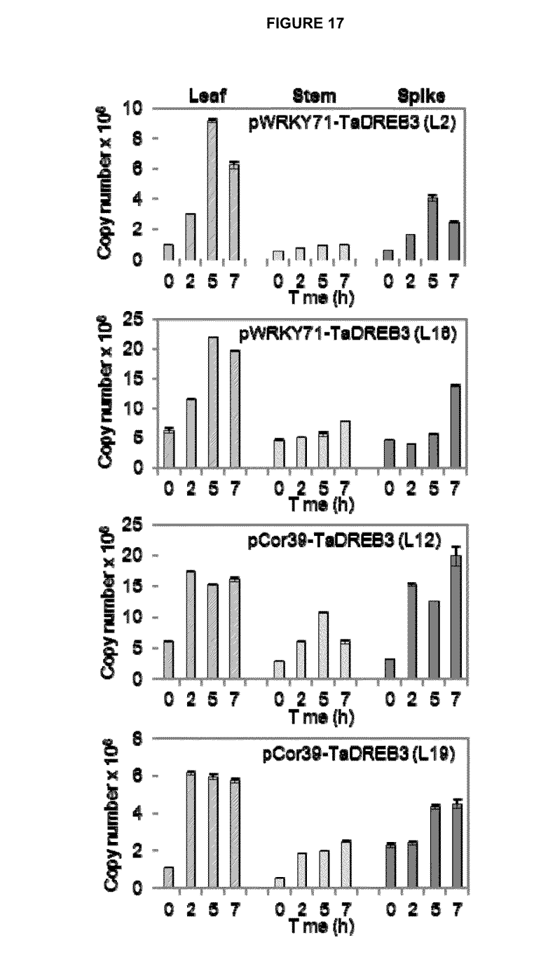

To assess tissue specific activation of the TdCor39 and OsWRKY71 promoters by cold, two lines for each construct (one flowering T₁ plant per line, all with a single copy of the transgene) were incubated at a constant 4° C., and tissue samples were collected at 0, 2, 5, and 7 hours of incubation. TaDREB3 expression was analysed by Q-PCR. As shown in FIG. 17, activation of both promoters by cold was observed in all tested tissues (leaf, stem and spike), although levels between basal and inducible transgene expression were very different. The OsWRKY71 promoter was activated in all tested tissues with higher levels in leaves than in spikes and stems. Activation of the promoter in stems after 7 hours of cold stress for both tested lines was less than two-fold over the constitutive level of promoter activity. In contrast, activation of the TdCor39 promoter over basal level of activity in stem was stronger than activation of the OsWRKY71 promoter.

Phenotypes of Transgenic Rice Transformed with the pWRKY71-TaDREB3 Construct and Activation of the OsWRKY71 Promoter in Rice

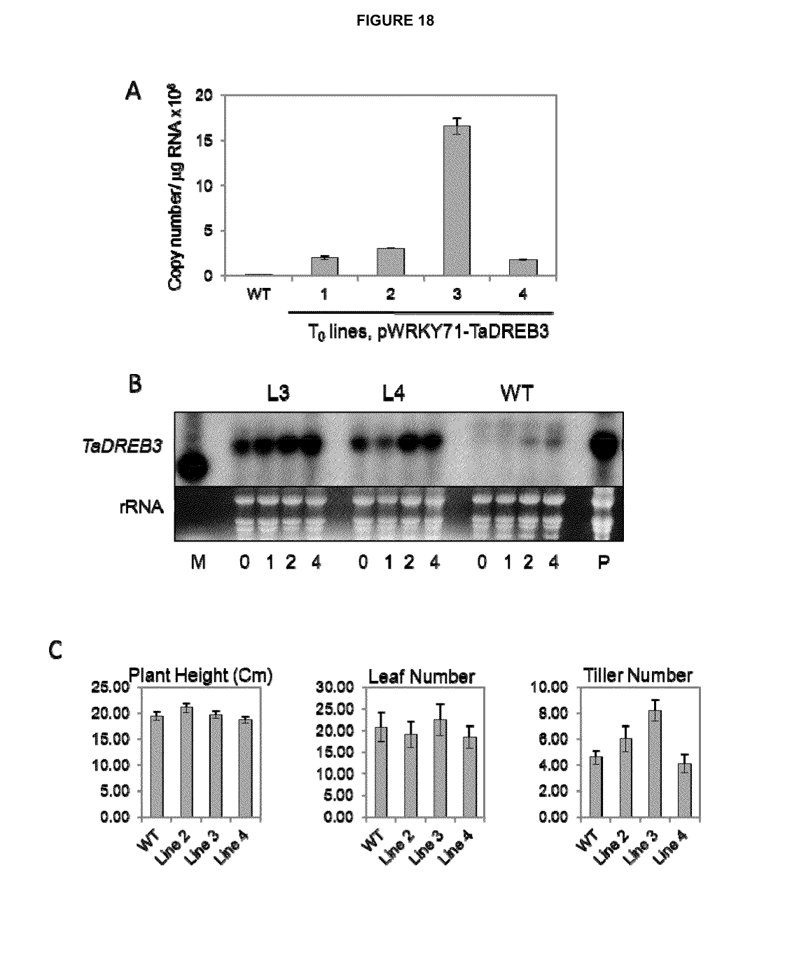

Three from four transgenenic rice lines with a single copy of the pWRKY71-TaDREB3 transgene were used for the analysis of plant phenotypes in the absence of stress. All transgenic rice lines showed a basal level of promoter activity, but which was highest in Line 3 (FIG. 18A). Induction of the promoter by cold was demonstrated in two lines using Northern blot hybridisation and a coding region of the transgene (TaDREB3) as a hybridisation probe (FIG. 18B). As shown in FIG. 18C, analysis of 5 to 10 T₁ plants for each line revealed no significant differences in plant height and leaf number compared to control (WT) plants. Line 4 produced the same number of tillers as control (WT) plants. Lines 2 and 3 developed more tillers.

### Discussion

Constitutive overexpression of stress-related transcription factors often provides improvement of plant survival under stress. However, strong constitutive overexpression of transcription factors is known to lead to the development of pleiotropic phenotypes in transgenic plants that often have a detrimental effect on yield. Accordingly, stress-inducible promoters were used to overcome this negative influence of transgene over-expression on plant development. However, the number of characterized cold-inducible promoters tested in grasses is low.

The wheat DREB3 gene was selected for the present study because it was previously demonstrated that constitutive overexpression of this gene in barley under the 2×35S promoter significantly improves frost tolerance of transgenic seedlings. However, its expression led to the development of a detrimental pleiotropic phenotype, the main features of which were stunted growth, delayed flowering and reduced grain yield (Morran et al. 2011, supra). The aim of this research was to decrease the pleiotropic effects of TaDREB3 overexpression on barley development, but improve frost tolerance by using stress-inducible promoters.

In this work we compared two promoters of very different cold-activated genes in combination with the same stress-tolerance gene, TaDREB3, in transgenic barley and rice plants. The activity of both OsWRKY71 and TdCor39 promoters was induced by several abiotic stresses and wounding. We demonstrate that the OsWRKY71 promoter was activated by cold in stems, leaves and spikes of transgenic barley (FIG. 17) and in leaves of transgenic rice (FIG. 18). It was also shown that during similar conditions of cold treatment the TdCor39 gene reached a maximum of expression at least several hours later than the OsWRKY71. Under treatment at a constant 4° C. both OsWRKY71 and TdCor39 genes remained active for at least 24 hours. Our data suggest that expression of both genes can also be induced by wounding and/or pathogens; however, in contrast to activation by abiotic stresses activation by wounding is rapid and not prolonged.

Our results demonstrate that the cold inducible promoters reduced the pleiotropic phenotype that was previously observed when DREB3 was constitutively expressed. In the case of the OsWRKY71 promoter the detrimental phenotype was strongly reduced and partial improvement was seen when the TdCor39 promoter was applied (FIG. 10). The negative influence of TaDREB3 expression on plant phenotypes seemed to correlate with the strength of promoter activity (quantified on the level of mRNA expression), which was higher overall for transgenic lines with the TdCor39 promoter and lower for transgenic lines with the OsWRKY71 promoter (FIG. 16). Most of the tested T₁ barley transgenic plants had a single copy of the transgene (FIG. 9A); however, levels of both constitutive and inducible expression were very different (FIG. 9B and FIG. 16). This difference in expression level suggests a strong influence is imposed by the position of the insertion event in the genomic DNA. Among the possible causes of such an influence could be control of the promoter activity by occasional distal enhancer/repressor elements and/or different accessibility of chromatin for trans-activators in diverse chromosome regions. Nevertheless, such variability of promoter strength allows for selecting lines without a penalty in yield in the “good” year, and increased vegetative frost tolerance in the year with extreme temperatures.

Another finding was the influence of the promoter on the initiation of a different number of tillers. The TaDREB3 driven by the OsWRKY71 promoter led to initiation of the same or an even larger number of tillers than in control plants, whereas use of the TdCor39 promoter lead to a significant decrease in tiller number (FIG. 10). This phenomenon can be explained by possible differences in the spatial patterns of constitutive components of the promoter. Reports from the literature suggest that the Cor39 gene may be active in tissues that are responsible for tiller initiation. Immunohistochemical localization of the WCS120-like proteins demonstrated their abundance in the vascular transition zone of crown meristematic tissue (Houde et al., 1995, Plant J. 8: 583-593), which is important for the re-growth of wheat seedlings after harsh winter conditions. However the question remains open, whether the expression of TaDREB3 under the TdCor39 promoter in shoot meristems and particularly in the vascular transition zone is the reason for the reduced or delayed initiation of tillers. The OsWRKY71 promoter might be not active or has weaker activity in the tissues responsible for tiller initiation and therefore cannot negatively influence tiller number. However, even in the case of the OsWRKY71 promoter, the tiller number and yield characteristics were lower and there was a longer delay in flowering in the progeny of Line 16, which had unusually strong transgene expression. There is also a simpler explanation for the difference in the number of tillers: the possibility of a delay in the development of plants with TaDREB3 driven by the TdCor39 promoter, which is supported by a relatively long delay in flowering of pCor39-TaDREB3 lines (FIG. 12). Because counting was done when control and transgenic plants were possibly at different stages of development, the lower tiller number was detected in transgenic plants. This explanation was supported by a comparable number of spikes in the control and transgenic plants at harvest (FIG. 10).

Both studied promoters were induced by cold in all experiments performed. However, the basal level of promoter activity was very different in different experiments and was obviously dependent on a stimuli not related to cold and dehydration. On the whole, the basal level of activity was lower in the glasshouse, when plants were grown in soil, and much higher in experiments on hydroponics (data not shown). The growth cabinet with different light quality from that of the glasshouse seems to have increased promoter activity in the absence of cold or drought. Weak induction of both promoters was detected 3 days after plants were transferred from the glasshouse to the growth cabinet (FIG. 10). The tissues were sampled exactly at the same time of the day to exclude the possible influence of circadian oscillations. Field trials are required to elucidate if this occasional promoter activation(s) can influence plant development and yield in the field.

Analysis of frost survival rates of 3-week-old transgenic barley seedlings was repeated several times for each promoter-TaDREB3 construct and transgene activation by cold was confirmed by Northern blot hybridization in all experiments. Transgenic seedlings with both promoter-TaDREB3 constructs demonstrated significantly better frost survival capacity than control plants (FIGS. 13, 14 and 15). Both the OsWRKY71 and TdCor39 promoters were activated by cold in all tested tissues including developing spikes (FIG. 17). Therefore, the best selected lines with good vegetative frost tolerance and minimal differences in flowering time and grain yield from control plants can be used in frost tolerance tests at flowering.

The expression of three cold-inducible LEA/COR/DHN genes, HvCor14b, HvDHN8 and HvA22, was studied by Q-PCR with the aim of confirming the presence of the functional TaDREB3 protein in transgenic plants. These three genes were earlier found to be up-regulated in response to constitutive over-expression of TaDREB3 (Morran et al., 2011, supra). Results showed that all three target genes were up-regulated by cold treatment to much higher levels in transgenic plants than they were in control plants (FIG. 16). This experiment revealed an unexpected result: the use of, as a whole, the weaker OsWRKY71 promoter provided stronger activation of downstream genes than the use of the more active TdCor39 promoter. There are at least three possible explanations for this phenomenon. Firstly, promoter sequences used in this work contained 5′-untranslated regions (5′UTRs) of respective genes that can potentially confer differences in the efficiency of translation, which in turn could lead to larger amounts of transgene product produced by “transcriptionally” weaker promoters. Secondly, the OsWRKY71 promoter in the same experimental conditions was activated earlier by cold than the TdCor39 promoter and hence, began to accumulate protein product earlier before the efficiency of protein synthesis declined as the result of prolonged treatment with low temperatures. Thirdly, the possible combination of two different spatial patterns of transgene expression and the presence/absence of modifying or modulating co-factors in particular tissues/cells can also influence expression of downstream genes, especially in the case of indirect transcriptional regulation. Stronger activation of the transgene in plants with the weaker OsWRKY71 promoter suggests that “transcriptional” strength of the promoter (transgene mRNA level) does not necessarily correlate with the amount of mRNA of downstream genes and if used as criteria for promoter selection should be used with caution.

To summarise, application of both cold-inducible promoters improved the developmental phenotypes of transgenic barley compared with constitutive expression. However, only the promoter of OsWRKY71 can be recommended to use for stress-inducible expression of DREB genes, which are encoding transcription factors that are usually in low abundance. The more powerful TdCor39 promoter, however, did not perform as well as the OsWRKY71 promoter in combination with a low abundant gene TaDREB3, but still might be a useful promoter for some highly abundant transcription factor genes, e.g. bZIPs, the function of which is dependent on ABA mediated modifications.

Those skilled in the art will appreciate that the invention described herein is susceptible to variations and modifications other than those specifically described. It is to be understood that the invention includes all such variations and modifications. The invention also includes all of the steps, features, compositions and compounds referred to, or indicated in this specification, individually or collectively, and any and all combinations of any two or more of the steps or features.

1. A method for effecting stress responsive expression of a nucleotide sequence of interest in one or more cells of a plant, the method including expressing in the one or more cells of the plant the nucleotide sequence of interest operably connected to a transcriptional control sequence which is stress inducible in the plant, wherein the nucleotide sequence of interest is heterologous with respect to the transcriptional control sequence, and wherein the transcriptional control sequence includes the nucleotide sequence set forth in SEQ ID NO: 1, or a functionally active fragment or variant thereof.

2. The method of claim 1, wherein the plant is a monocot plant, a cereal crop plant, or a wheat, rice or barley plant.

3-5. (canceled)

6. The method of claim 1, wherein the nucleotide sequence of interest includes a nucleotide sequence which, when expressed by one or more cells of a plant, improves the stress tolerance of the plant.

7. The method of claim 6, wherein expression of the nucleotide sequence does not disturb development of the plant.

8. The method of claim 1, wherein the stress is cold.

9-10. (canceled)

11. The method of claim 1, wherein the nucleotide sequence of interest includes a nucleotide sequence that encodes a DREB polypeptide.

12. The method of claim 11, wherein the DREB polypeptide is a TaDREB3-like polypeptide.

13. A method for improving the cold tolerance of a plant, the method including expressing a nucleotide sequence of interest in one or more cells of the plant according to the method of claim 8.

14. A nucleic acid construct including a nucleotide sequence of interest operably connected to a transcriptional control sequence which is stress inducible in a plant, wherein the nucleotide sequence of interest is heterologous with respect to the transcriptional control sequence, wherein the transcriptional control sequence includes the nucleotide sequence set forth in SEQ ID NO: 1, or a functionally active fragment or variant thereof.

15. The nucleic acid construct of claim 14, wherein the plant is a monocot plant, a cereal crop plant, or a wheat, rice or barley plant.

16-18. (canceled)

19. The nucleic acid construct of claim 14, wherein the nucleotide sequence of interest includes a nucleotide sequence which, when expressed by one or more cells of a plant, improves the stress tolerance of the plant.

20. The nucleic acid construct of claim 19, wherein expression of the nucleotide sequence does not disturb development of the plant.

21. The nucleic acid construct of claim 14, wherein the stress is cold.

22-23. (canceled)

24. The nucleic acid construct of claim 14, wherein the nucleotide sequence of interest includes a nucleotide sequence that encodes a DREB polypeptide.

25. The nucleic acid construct of claim 24, wherein the DREB polypeptide is a TaDREB3-like polypeptide.

26-27. (canceled)

28. A genetically modified cell including the nucleic acid construct of claim 14, or a genomically integrated form thereof.

29. The cell of claim 28, wherein the cell is a plant cell, a monocot plant cell, a cereal crop plant cell, or a wheat, rice or barley plant cell.

30-32. (canceled)

33. A multicellular structure including one or more cells of claim 28.

34. The multicellular structure of claim 33, wherein the multicellular structure includes a plant or a part, organ or tissue thereof, and wherein a nucleotide sequence of interest is expressed in one or more cells of the plant or a part, organ or tissue thereof in response to stress.

35-38. (canceled)

39. The multicellular structure of claim 34, wherein the plant or a part, organ or tissue thereof has improved stress tolerance relative to a plant or a part, organ or tissue thereof which does not include the one or more cells.

40. (canceled)


SEQUENCE LISTING (31 sequences)

SEQ ID NO: 1 - DNA, 2331 bp, Oryza sativa
caccagacgttccaatcctaataaccctacattggagtagtacaagaactggaaaatttgggatcatttgcataattaagcttagtaaacgacccaacggcgcccgccggtcaataacagtggattaatcatgctattagttgtcataaacaaactttttattttggatgcagtattgactcgtgcggcgagagtagtcttccccaaaagtatttctagtcaaaatttggcaaaaacctaagctattgtatagtacaaaaagctctttccggatgtttgatttgatttaatttaagaggttaatctagggcccctcaagtttggagctcaccaaaccaggaaagacaccaagaaaacatagtactgtactaactatgtgcatcacacctaccctgctctacaagatactctagtactatagctactttgcccagttgcactaggaattaagcataggggggaaatgaaatcggaacgggagtcaaaaaaaaaaaaaaagcaacctaggagcgaatctggacaaacctctgtctagattcgtggtagtaggagggtcacatcccttcctacattgcttattttgggacagagggagtacaccatccacaaatatgtgagattttaggttattttagcaaaatttaaggttaagtggaaattaattgatgcctcagataatttacttgattagaaaaatcacgcctaatgatttaactaccagtgaataataaatgtagctattgtatttgcaaacaaaatttaaaacctgaaaatgtttatattcgtgtatggagagagtgtttttctaataaaacacataaagccaagaaaatgagtttctagtagagttgaaaaggggaagaattaagaggttagacactgatccagctaagttattattggcaagtgcatgctcactgacaagcaagggtccaaaaggtggtactccttccgttttgaaatatttgacgccgttgactttttagcacatgtttaaccgttcgtcttattcaaaaaatttaaataattattaattcttttcctatcatttgattcattgttaaatatatttttatataggcatataactttacatattttacagaagtttttaaataagacaaatggtcaaacatatgctaaaaaatcaatggtgttaaatattttgaaacgggggagtatttagtagtagggggtcaagaagtcttgcatgtccccttcttcctccaacttcttctttgccaattgcaaggcatgcagatagggatctttattaatagcaggcatgtgtggtgtattttcttgactttggaccctgccccactatccagcccaaagaccatgcatcatgctgctggccgacatcctgactagtctcccacccactcgttattacatcccctaattaattaatcatttctattttgacaagataaaccatacctttctctaaaatatagagatttctaatatttatactttggactatttggtagagtttcaactcctaaatttagcttcagaagtaattaagtctaaattagaattgtgaagcaacctaaacctagttccaactcttaattttattttgtgagagagctccacccaactctatttctattttaggtggagctgaaattgtttggttgagctggagctgtgccaaacaggctctttatctaaaatatagctatttcttcttgtgctatgtcatatcaaacaattataacaatgttttatttaatttatctctatatattttatttcaaataatcataaccatcttttgttaaattataaaacgtcttatccactcaaaaaccctagtaatattttaaaacgagtattatgaaatgacgagatgacaaacacaagaactagagggatgtggtttttatttttgttttaaaaaataataattcagttggcgccgctagctgttccggtcaccctccagtctccttccgattcttccgccacatccgcatgctcgaattccccctccctccatttctttactcatttcccttcccctccgcttcctaatcctgcgtgaccccagacgacgacgacgacgagtacacatgcgtttgagcgcgacatccccgcgcgcgccttcttcttcctcttcctccctcctcccatgactagaccatcctcgccatattcccgcctctctatatatattagggctcagctcctcctctcacctcgtctcctcctctcctctcttctctccttggctcagcttgacatcgtcggagcagcagaaaagtttgctcgatcgagctagctaactaggtcgtcggatcagtgataaatcgttcgccg

SEQ ID NO: 2 - DNA, 27 bp, Artificial Sequence
  OsWRKY71F primer nucleotide sequence - promoter amplification
gccaagcttcttagtaaacgacccaac

SEQ ID NO: 3 - DNA, 27 bp, Artificial Sequence
  OsWRKY71R primer nucleotide sequence - promoter amplification
cccggtacccggcgaacgatttatcac

SEQ ID NO: 4 - DNA, 21 bp, Artificial Sequence
  TaCor39F primer sequence - TdCor39 amplification
atggagaaccaggcacacatc

SEQ ID NO: 5 - DNA, 20 bp, Artificial Sequence
  TaCor39R primer sequence - TdCor39 amplification
ggtcattccagtgtgtgcat

SEQ ID NO: 6 - DNA, 2207 bp, Triticum durum
ttggagtgggcaaagacgatgtggcttaacagaggtcagtgcaggatttgaagaagatggaagaagaacaagaagcagcaggagcagtagcagaagcagaagtagaagaagaggagaggaagagaaagatctggtagcaccgatgaactagcccctatttagctaatggctcttaataatttgaactgtcatttgatagtagttaggatgaaccgtcatttgtttaagtagatggcgctacatattcagattctgtgtggcaagctcaccactagttagaagtgatgacaacaccttatgcaggttgcaaccaaacaccatgtcatatgtgcgcctaatgcaatgcgggcaaccaaacgccgggtcgaaaatggttatctcatgcaactaagggcatgcaggcaatcaaactatgtgcatctgattttttttggcctgcatcccctcaaaccggctcaatagagccagcctcgccggaccaggctgaatcagcaatgtaaccaaacacgccctagtgattcaacatatttctaactcatatttttagaataacagggagtgagctgagttccccggctccacaacagcgttttaggacagacccccccccccccccccctaatgtgcttcgatcccactgtttttcctacgcgtggctgcagtttaccatccagcaacggctcatgtggttgcgtcgcgcttgaatcataccgcctctgatgataactgaaagtctgaaattaccataggtcaagtgcgagtgtaggatgaaaaaagtgtctataaattgcttcaatatttttgttgtgctttgtttatatagaaagcaagtcatatgctgctctgatatgagattattgcataaacatattaactttttttatcttaaaattatatctatgtatggaggtgctactgttgctggcggcagcacttccagttttcagttgagaccagataattgtgatcgatttcaccccctcttttgggtcatctgttcttacttctgctttggctcaccagcattcatctagtgtgaatccccaacctgcatttaaatactgcactgaatatctatagtctagcaaatcaagtttgtttgatgcaaaaattctgaggatgttgctctctgctattgtcttcgcaaattgcctgcttcacggttttgacttcattgtggttgtgtttctttactgctaaaatacgaaggattgtagacatgtcctgaatcctatgaattagaaaactacatgagttcgaataaaagattgtttgatattatccctataaaagcaaaggaagtctacaaagatatttcagtggatgcgcatttttctccagaataaagtgcaaatgaattttaaggaaaattactaaacatttcaatcctataaatcaaacagcaacatagagtgtacatttggcttcaaaatttgtccacaaaagagtgtacttctatcttctcaatgcactttaaagtagaaaaacatacttctctctcatcacatggtaatcaagacaagtaacaatctacacatgatctttttaatttctacatacacttagctcattagggttggataattaaagatgagagagatgatggcttgcatctttccaatacattttttacttcacttcataatttgttttaaaatctctaaggacaaattaccggacggagagagtaaatcgtctaaaaatcctataataatttcttttgaatcaaagcccttagcttctcgtatgcttcgtcgttacatgccgacactttggatcttccatcctcttatcttaagcaaacaataccatttttgcaagcgaaaagaaggatcagatttttcggggaatcatcttcttcccggccggacgaggacgaatgagctgggacgtggcgcccgggacgcgccaactagcttaagaggcccggcccctctagtcggcagccatctgccgaccaccgatgccaccgcacgtagcgcccagccgcggcgattcgtccatctgaccacccctctttatgggctagtcggcactcacctgcccgtccactcacgaccgcgcacgtcgtgcttcgtctaccctccaccggcctataaatactggcccgtgctgcacatactttgcacaaccacctgcttcacacagccaagagaagtaaacacatcagcactagtagatttcacgagtcagaagctcagcgcaag

SEQ ID NO: 7 - DNA, 21 bp, Artificial Sequence
  TdCor39F primer sequence - promoter amplification
cacctgttacaagatagcatc

SEQ ID NO: 8 - DNA, 21 bp, Artificial Sequence
  TdCor39R primer sequence - promoter amplification
cttgcgctgagcttctgactc

SEQ ID NO: 9 - DNA, 21 bp, Artificial Sequence
  PF3′ primer sequence
gtatctcgcatatggacggag

SEQ ID NO: 10 - DNA, 21 bp, Artificial Sequence
  NTR5′ primer sequence
ttgccaaatgtttgaacgatc

SEQ ID NO: 11 - DNA, 20 bp, Artificial Sequence
  HPF primer sequence - Q-PCR normalisation (barley and wheat)
attttccagtcacctggccc

SEQ ID NO: 12 - DNA, 19 bp, Artificial Sequence
  HPR primer sequence - Q-PCR normalisation (barley and wheat)
tgctatctggctcagctgc

SEQ ID NO: 13 - DNA, 21 bp, Artificial Sequence
  TMBW probe sequence - TaqMan probe (barley and wheat)
atggtggaagggcggctgtga

SEQ ID NO: 14 - DNA, 18 bp, Artificial Sequence
  SPSF primer sequence - Q-PCR normalisation (rice)
ttgcgcctgaacggatat

SEQ ID NO: 15 - DNA, 20 bp, Artificial Sequence
  SPSR primer sequence - Q-PCR normalisation (rice)
cggttgatcttttcgggatg

SEQ ID NO: 16 - DNA, 19 bp, Artificial Sequence
  TMR probe sequence - TaqMan probe (rice)
gacgcacggacggctcgga

SEQ ID NO: 17 - DNA, 20 bp, Artificial Sequence
  HygF primer sequence - Q-PCR transgene analysis
cgctcgtctggctaagatcg

SEQ ID NO: 18 - DNA, 20 bp, Artificial Sequence
  HygR primer sequence - Q-PCR transgene analysis
agggtgtcacgttgcaagac

SEQ ID NO: 19 - DNA, 24 bp, Artificial Sequence
  TMTr probe sequence - TaqMan probe (transgene)
tgcctgaaaccgaactgcccgctg

SEQ ID NO: 20 - DNA, 21 bp, Artificial Sequence
  TaDREB3F primer sequence - endogene Q-PCR
ctcgattcgcttgctcctcag

SEQ ID NO: 21 - DNA, 24 bp, Artificial Sequence
  TaDREB3R primer sequence - endogene Q-PCR
tcctgatgacaagctgtagtgtgc

SEQ ID NO: 22 - DNA, 21 bp, Artificial Sequence
  TxDREB3F primer sequence - transgene Q-PCR
gtatctcgcatatggacggag

SEQ ID NO: 23 - DNA, 21 bp, Artificial Sequence
  TxDREB3R primer sequence - transgene Q-PCR
ttgccaaatgtttgaacgatc

SEQ ID NO: 24 - DNA, 21 bp, Artificial Sequence
  HvCor14BF primer sequence - cold-responsive Q-PCR
ttgaggatgtgagcaaatgag

SEQ ID NO: 25 - DNA, 21 bp, Artificial Sequence
  HvCor14BR primer sequence - cold-responsive Q-PCR
tacatcgtcaatgacgagacc

SEQ ID NO: 26 - DNA, 18 bp, Artificial Sequence
  HvDhn8F primer sequence - cold-responsive Q-PCR
gcgagcacaagcccagag

SEQ ID NO: 27 - DNA, 23 bp, Artificial Sequence
  HvDhn8R primer sequence - cold-responsive Q-PCR
gcccagcaataaaccaatacaca

SEQ ID NO: 28 - DNA, 21 bp, Artificial Sequence
  HvDhn5F primer sequence - cold-responsive Q-PCR
tggcgaagttccaccgtatgc

SEQ ID NO: 29 - DNA, 21 bp, Artificial Sequence
  HvDhn5R primer sequence - cold-responsive Q-PCR
acgaaaactgttgccacactg

SEQ ID NO: 30 - DNA, 22 bp, Artificial Sequence
  HvA22F primer sequence - cold-responsive Q-PCR
gctcctcacccacctccactcc

SEQ ID NO: 31 - DNA, 23 bp, Artificial Sequence
  HvA22R primer sequence - cold-responsive Q-PCR
ctgagctgctccctgacgacctt